SEQUENCE LISTING (21 sequences)

SEQ ID NO: 1 - DNA, 37 bp, Synthetic construct
  source 1..37: mol_type = other DNA
atcggtggtctccggagtttcgctatacagagcagaa

SEQ ID NO: 2 - DNA, 40 bp, Synthetic construct
  source 1..40: mol_type = other DNA
atcggtggtctccaactaaaccacagtgaaatttttaaga

SEQ ID NO: 3 - DNA, 33 bp, Synthetic construct
  source 1..33: mol_type = other DNA
atcggtggtctcgttgtgaacattgcagacctg

SEQ ID NO: 4 - DNA, 35 bp, Synthetic construct
  source 1..35: mol_type = other DNA
atcggtggtctcgatggatatcggcccttgctgtc

SEQ ID NO: 5 - DNA, 37 bp, Synthetic construct
  source 1..37: mol_type = other DNA
atcggtggtctccagtttatgttgctgtacttggatc

SEQ ID NO: 6 - DNA, 43 bp, Synthetic construct
  source 1..43: mol_type = other DNA
atcggtggtctccacaaaaatgtgactattaattttgactttt

SEQ ID NO: 7 - DNA, 21 bp, Synthetic construct
  source 1..21: mol_type = other DNA
aagcaaatgctttaggtgcat

SEQ ID NO: 8 - DNA, 20 bp, Synthetic construct
  source 1..20: mol_type = other DNA
tcggaggtagcgtgtcctta

SEQ ID NO: 9 - DNA, 20 bp, Synthetic construct
  source 1..20: mol_type = other DNA
tccgcaggatgtttcacata

SEQ ID NO: 10 - DNA, 20 bp, Synthetic construct
  source 1..20: mol_type = other DNA
gcatgggcgtgggtttttag

SEQ ID NO: 11 - DNA, 20 bp, Synthetic construct
  source 1..20: mol_type = other DNA
tcggaggtagcgtgtcctta

SEQ ID NO: 12 - DNA, 20 bp, Synthetic construct
  source 1..20: mol_type = other DNA
gcatgggcgtgggtttttag

SEQ ID NO: 13 - DNA, 17 bp, Synthetic construct
  source 1..17: mol_type = other DNA
ctcagaacgaacgctgg

SEQ ID NO: 14 - DNA, 19 bp, Synthetic construct
  source 1..19: mol_type = other DNA
catttctagtggctatccc

SEQ ID NO: 15 - DNA, 22 bp, Synthetic construct
  source 1..22: mol_type = other DNA
cgcagcttgctgcgtgtatggt

SEQ ID NO: 16 - DNA, 18 bp, Synthetic construct
  source 1..18: mol_type = other DNA
cgcgtgggatattctttc

SEQ ID NO: 17 - DNA, 19 bp, Synthetic construct
  source 1..19: mol_type = other DNA
ccgggaaagacagtttaag

SEQ ID NO: 18 - DNA, 24 bp, Synthetic construct
  source 1..24: mol_type = other DNA
agcctatgtgaaacatcctgcgga

SEQ ID NO: 19 - DNA, 20 bp, Synthetic construct
  source 1..20: mol_type = other DNA
acaatctctcggcaggcaaa

SEQ ID NO: 20 - DNA, 20 bp, Synthetic construct
  source 1..20: mol_type = other DNA
cggtcatggaatctcgcctt

SEQ ID NO: 21 - protein, 184 aa, Anaplasma marginale
MLANPAFHVVRKAPPTHFPVALTEVKSFLGINNSSEDSLLTSIISMSSEYAQWYIGQSLGRQTWVLSYSRGNIPKKIYLPFGPVVSVVSVSVATQNTSLRSVTRKEYSIDNLQSSVVHAAIGATKIEIVYEAGYADMRDVPTQIKHGILHHVAVAYKRRDSMTVEHLAFIKEIYMPFREVRLVL

(19) United States
(12) Patent Application Publication
(10) Pub. No.: US 2025/0161426 A1
(43) Pub. Date: May 22, 2025

(54) ANAPLASMA VACCINES AND METHODS OF USE THEREOF

(71) Applicant: Kansas State University Research Foundation, Manhattan, KS (US)

(72) Inventor: Roman R. Ganta, Manhattan, KS (US)

(21) Appl. No.: 18/841,203

(22) PCT Filed: Feb. 23, 2023
(86) PCT No.: PCT/US2023/063179

§ 371 Date: Aug. 23, 2024

Related U.S. Application Data
(60) Provisional application No. 63/268,473, filed on Feb. 24, 2022.

(51) Int. Cl. A61K 39/02; A61K 39/00

(52) U.S. Cl. CPC A61K 39/0233; A61K 2039/521; A61K 2039/522; A61K 2039/552; A61K 2039/55566

(57) ABSTRACT

The present disclosure provides an immunogenic composition against A. marginale wherein the composition generally includes a disrupted or deleted phtcp gene. Such compositions are useful in reducing the incidence, severity, transmission, and duration of infection with A. marginale.

### RELATED APPLICATIONS

The present application claims the priority benefit of provisional application Ser. No. 63/268,473, filed on Feb. 24, 2022, the teachings and content of which are incorporated by reference herein.

### GOVERNMENT RIGHTS STATEMENT

This invention was made with government support under PHS grant #s AI070908 and AI152418 awarded by the National Institute of Allergy and Infectious Diseases, National Institutes of Health, USA. The government has certain rights in the invention.

### SEQUENCE LISTING

This application contains a sequence listing in paper format and in computer readable format, the teachings and content of which are hereby incorporated by reference.

### BACKGROUND OF THE DISCLOSURE

Tick-borne Anaplasma spp. are obligate, intracellular, tick borne, bacterial pathogens that cause important diseases globally in people, agricultural animals, and dogs. Over the last few decades, several new, emerging and reemerging tick-borne rickettsial diseases have been identified as a major public health concern in the US and globally. Similarly, tick-borne diseases are responsible for major economic losses in food and agricultural animal production throughout the world. The much-needed targeted mutagenesis methods are yet to be fully developed and are of great importance for defining genes essential for several obligate intracellular bacterial pathogens, including the Anaplasmataceae transmitted by ticks. Notably, the lack of targeted mutagenesis methods is regarded as a major obstacle for advancing research in defining the pathogenesis of obligate intracellular bacteria and developing effective interventions, including vaccines. Mutagenesis methods can aid in developing efficacious vaccines against such pathogenic bacteria. However, the disruption and successful restoration of gene function is not straightforward nor easy.

Anaplasma marginale (“A. marginale”) infects bovine erythrocytes and causes anemia, abortion, poor production, and can also cause high mortalities in beef and dairy cattle globally. Despite the estimated major global economic losses due to bovine anaplasmosis amounting to billions of US dollars annually, efficacious and safe vaccines for worldwide use in controlling the disease are currently not available. Although a heterologous A. centrale-based live blood vaccine is used as a means of reducing disease severity caused by A. marginale in parts of sub-Saharan Africa, Israel, Uruguay, and Australia, it is not in use in other parts of the world, including Europe and North America as it may result in introducing other blood-borne diseases. While an experimental killed vaccine is marketed by the Louisiana State University Agricultural Center, there are no scientific reports describing its production and efficacy. Additionally, development of subunit vaccines against bovine anaplasmosis has been met with limited success. A recent study reported the feasibility of developing a live attenuated vaccine using an A. marginale strain with a random insertion mutation, though the work has yet to progress beyond the initial description. Given the insufficiencies of the current control options, coupled with the high economic burden associated with bovine anaplasmosis, investigations focused on developing alternative vaccines for the disease control remain a high-priority goal.

### SUMMARY OF THE DISCLOSURE

The present disclosure overcomes the problems inherent in the prior art and in one aspect provides a targeted mutagenesis method for Anaplasma marginale. In some forms, targeted mutagenesis is utilized to produce at least one disrupted or deleted gene in A. marginale to produce a mutant A. marginale. In some forms, the gene is the phtcp gene. In some forms, the mutant A. marginale is part of an immunogenic composition useful against A. marginale infection. In some forms of the mutated A. marginale, the phage head-to-tail connector protein gene is deleted. In such forms, the mutant does not cause disease and exhibits attenuated growth in its natural host (cattle). It is believed that the functional disruption and/or deletion in the A. marginale phtcp gene is likely detrimental in altering the pathogen's ability to obtain metal ions to support its growth within a phagosome of infected host macrophages. This functional disruption in the gene encoding the membrane-bound phage head-to-tail connector protein (phtcp) causes rapid pathogen clearance from a host, while inducing sufficient immune response in conferring protection against wild-type infection challenge by both intravenous inoculation and by tick transmission.

In other forms, the present disclosure provides an immunogenic composition that confers protection against wild-type A. marginale infection challenge. In some forms, the protection is evidenced by a decrease in the severity, incidence of, and/or duration of at least one sign of infection by or with A. marginale. In some forms, the evidence of protection is a decrease in the transmissibility of A. marginale. In some forms, the evidence of protection is a decrease in the growth of A. marginale in a host or susceptible animal. In some forms, the decrease is in an individual animal or a group of animals susceptible to A. marginale infection. In some forms, the decrease is in comparison to an animal or group of animals that has not received at least one administration of an immunogenic composition of the present disclosure. In some forms, the sign of infection is selected from the group consisting of anemia, fever, abortion, pale mucous membranes, jaundice, weight loss, poor production, decrease in weight gain, death, anaplasmosis, weakness, pallor, lethargy, dehydration, anorexia, pale tissues, decreased packed cell volume, watery blood, thin blood, splenomegaly, hepatomegaly, gall bladder distension, membrane-bound inclusions (colonies) in the cytoplasm of infected erythrocytes, loss of megakaryocytes in bone marrow, adipocyte atrophy, cholesterol clefts, edema, and hemolysis. In some forms, the decrease in the severity, incidence of, duration of at least one sign of A. marginale infection is at least 10, 20, 30, 40, 50, 60, 70, 75, 80, 85, 90, 91, 92, 93, 94, 95, 96, 97, 98, 99, or even 100%. In some forms, the decrease in transmissibility of A. marginale is from a host animal to a vector. In some forms, the decrease in transmissibility is at least 10, 20, 30, 40, 50, 60, 70, 75, 80, 85, 90, 91, 92, 93, 94, 95, 96, 97, 98, 99, or even 100%. In some forms, the vector is a tick. In some forms, the tick is selected from the group consisting of an ixodid tick. In some forms, the tick is selected from the group consisting of D. andersoni, D. occidentalis, D. variabilis, R. sanguineus, R. simus, R. bursa, B. microplus, B. annulatus, B. decoloratus, and I. ricinus. In some forms, the vector is a blood-sucking fly from the family Tabanidae (horse flies and deer flies) or a mosquito. In some forms, the animal is a ruminant. In some forms, the ruminant is a cow, sheep, goat, or deer. In some forms, the immunogenic composition of the disclosure is administered at least one time to the animal in need thereof. When the immunogenic composition is administered more than one time, it is preferred to wait at least 2, 3, 4, 5, 6, 7, 8, or more weeks between subsequent administrations.

In some forms, the immunogenic composition includes whole cell A. marginale inactivated antigens as a vaccine (WCAV) candidate. Upon infection challenge, non-vaccinated control cattle developed severe disease with an average 57% drop in packed cell volume (PCV) between days 26-31 post infection with infection peaking to about 11% in erythrocytes and exhibiting anisocytosis. Conversely, following challenge, all animals receiving the live mutant composition according to the disclosure didn't develop clinical signs, anemia and lacked infected erythrocytes. In contrast, the WCAV vaccinees developed similar disease as in the non-vaccinees receiving A. marginale infection, however, the peak erythrocyte infection reduced to 6% and the PCV drop was reduced to 43%.

In some forms, the immunogenic composition comprises a complete gene deletion mutation in Anaplasma marginale, the pathogen responsible for causing severe economically important disease in cattle and other ruminants throughout the world. In some forms, the immunogenic composition comprising the gene deletion limits growth of A. marginale in a susceptible animal. In some forms, the gene deletion is accomplished through a targeted gene deletion method. In some forms, the deleted gene is the phtcp gene.

In some forms, the immunogenic composition comprises a gene disruption in Anaplasma marginale, the pathogen responsible for causing severe economically important disease in cattle and other ruminants throughout the world. In some forms, the immunogenic composition comprising the gene disruption limits growth of A. marginale in a susceptible animal. In some forms, the gene disruption is accomplished through a targeted gene deletion method. In some forms, the disrupted or deleted gene is the phtcp gene. In some forms, the disruption of the phtcp gene results in a lack of expression of at least a portion of this gene. In some forms, less than 90, 80, 70, 60, 50, 40, 30, 20, 10 or even 5% of the phtcp gene is expressed. In some forms, no portion of the gene is expressed. In some forms, the phtcp gene has at least 80, 85, 90, 91, 92, 93, 94, 95, 96, 97, 98, 99, or even 100% sequence homology or sequence identity with the phtcp gene from the St. Maries strain, GenBank #CP000030. In some forms, the phtcp gene will encode a protein having at least 80, 85, 90, 91, 92, 93, 94, 95, 96, 97, 98, 99, or even 100% sequence homology or sequence identity with SEQ ID NO. 21.

In some forms, the present disclosure provides a method of reducing the severity, incidence of, or duration of clinical disease or blood abnormalities caused by A. marginale infection. In some forms, the method reduces the transmissibility of A. marginale, preferably from a host animal to a vector. In some forms, the method includes the steps of administering an immunogenic composition of the disclosure to an animal in need thereof. In some forms, the immunogenic composition is administered once and in other forms, the immunogenic composition is administered two, three, four, or more times, preferably separated in time by at least 2, 3, 4, 5, 6, 7, 8 weeks, or more between subsequent administrations.

In the current disclosure, a targeted mutagenesis method for an Anaplasma sp. was developed. In general, the method deleted the phtcp gene from the A. marginale genome to form a mutant pathogen. In some forms, cattle receiving infection with the mutant exhibited no clinical disease and that the mutant pathogen had in vivo growth defect. In other forms, prior infection with the mutant offered protection against the virulent disease caused by the wild-type A. marginale and also kept the infected erythrocytes in animals to below detectable levels by microscopy. In still other forms, an A. marginale whole cell antigen-based inactivated vaccine (WCAV) was tested and it failed to prevent clinical disease following wild-type infection challenge. However, the WCAV was able to reduce peak erythrocyte infection to 6% and PCV drop to 43%, each of which are advantageous. The current disclosure represents the first description of targeted mutagenesis in an Anaplasma sp. and illustrates its application in defining a gene essential for in vivo growth and the development of a live attenuated vaccine to protect cattle from severe anaplasmosis and other signs of infection by A. marginale.

In some forms, the immunogenic composition or vaccine may include at least one further antigen from a pathogen other than A. marginale, making it a combination vaccine or immunogenic composition. In such an embodiment, an effective amount of a vaccine or immunogenic composition administered provides effective protection including a reduction in the severity or incidence of clinical signs of infection up to and including immunity against infections caused by A. marginale bacteria and at least one further disease-causing organism. The further pathogen is preferably selected from the group consisting of: Actinobacillus pleuropneumonia; Adenovirus; Alphavirus such as Eastern equine encephalomyelitis viruses; Bordetella bronchiseptica; Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl. septicum, Cl. tetani; Coronavirus, preferably Porcine Respiratory Corona virus; Eperythrozoonosis suis; Erysipelothrix rhusiopathiae; Escherichia coli; Haemophilus parasuis, preferably subtypes 1, 7 and 14: Hemagglutinating encephalomyelitis virus; Japanese Encephalitis Virus; Lawsonia intracellularis; Leptospira spp.; preferably Leptospira australis; Leptospira canicola; Leptospira grippotyphosa, Leptospira icterohaemorrhagicae; and Leptospira interrogans; Leptospira pomona, Leptospira tarassovi; Mycobacterium spp. preferably M. avium; M. intracellulare; and M. bovis; Mycoplasma hyopneumoniae (M hyo); Pasteurella multocida; Porcine cytomegalovirus; Porcine Parvovirus; Porcine Reproductive and Respiratory Syndrome (PRRS) Virus; Porcine circovirus, Pseudorabies virus; Rotavirus; Salmonella spp.; preferably S. thyhimurium; and S. choleraesuis; Staph. hyicus; Staphylococcus spp., Streptococcus spp., preferably Strep. suis; Swine herpes virus; Swine Influenza Virus; Swine pox virus; Swine pox virus; Vesicular stomatitis virus; Virus of vesicular exanthema of swine; Leptospira hardjo; Mycoplasma hyosynoviae; Poliovirus; Rhinovirus; hepatitis A virus; foot-and-mouth disease virus (FMDV); swine vesicular disease (SVDV), and combinations thereof.

The immunogenic compositions can further include one or more other immunomodulatory agents such as, e. g., interleukins, interferons, or other cytokines.

The immunogenic compositions can also include Gentamicin and Merthiolate.

While the amounts and concentrations of adjuvants and additives useful in the context of the present invention can readily be determined by the skilled artisan, the present invention contemplates compositions comprising from about 50 μg to about 2000 μg of adjuvant. Thus, the immunogenic composition as used herein also refers to a composition that comprises from about lug/ml to about 60 μg/ml of antibiotics or immunomodulatory agents, and more preferably less than about 30 μg/ml of antibiotics or immunomodulatory agents.

According to a further aspect, at least one further administration of at least one dose of the immunogenic composition as described above is given to a subject in need thereof, wherein the second or any further administration is given at least 7 days beyond the initial or any former administrations. Preferably, the immunogenic composition is administered with an immune stimulant. Preferably, said immune stimulant is given at least twice. Preferably, at least 3 days, more preferably at least 5 days, even more preferably at least 7 days are in between the first and the second or any further administration of the immune stimulant. Preferably, the immune stimulant is given at least 10 days, preferably 15 days, even more preferably 20, even more preferably at least 22 days beyond the initial administration of the immunogenic composition provided herein. A preferred immune stimulant is, for example, keyhole limpet hemocyanin (KLH), preferably emulsified with incomplete Freund's adjuvant (KLH/ICFA). However, it is herewith understood, that any other immune stimulant known to a person skilled in the art can also be used. The term “immune stimulant” as used herein, means any agent or composition that can trigger the immune response, preferably without initiating or increasing a specific immune response, for example the immune response against a specific pathogen. It is further instructed to administer the immune stimulant in a suitable dose.

In further aspect, the present disclosure provides methods for generating at least one stable targeted mutation by allelic exchange for the genera Anaplasma.

In a still further aspect of the present disclosure, Anaplasma mutated using the disclosed methods are useful in immunogenic compositions against Anaplasma pathogens. In preferred forms, such immunogenic compositions are effective at reducing the incidence or severity of at least one clinical sign of infection caused by or associated with infection by such pathogens. In some preferred forms, such compositions are administered prophylactically such that clinical signs are reduced in incidence and/or severity. In particularly preferred forms, the clinical signs are prevented in animals receiving such compositions prior to being infected or challenged with Anaplasma pathogens. In other forms, such compositions are administered after infection by Anaplasma pathogens has already occurred. In such situations, the clinical signs associated with or caused by the infections are reduced in incidence, severity, and/or longevity.

In a further aspect, the stable targeted mutation disrupts the function of at least one gene.

In yet a further aspect, the function of a gene that has been disrupted can be restored.

In yet a further aspect, a method for eliciting an immune response in a human or animal is provided, where the steps include administration of an immunogenic composition or vaccine disclosed herein to an animal or human in need thereof. Additionally, a method for reducing the incidence and/or severity of at least one or more clinical signs associated with or caused by infection by a pathogen from the genera Anaplasma is provided. In some forms, the infection is caused by or associated with A. marginale and the immunogenic composition or vaccine comprises at least one of those species wherein the targeted gene disruption or mutation is performed on such a species. Such a method generally comprises the step of administering the immunogenic composition to an animal in need thereof to elicit an immune response against infection and subsequent exhibition of clinical signs in animals infected or challenged by a species of the genera Anaplasma after administration of the immunogenic composition. In some forms, the immunogenic composition includes a modified live species of Anaplasma.

The immunogenic composition according to the disclosure may be administered intravenously, intramuscularly, intranasally, intradermally, intratracheally, intravaginally, intravenously, intravascularly, intraarterially, intraperitoneally, orally, intrathecally, or by direct injection into any target tissue. Depending on the desired duration and effectiveness of the treatment, the immunogenic compositions according to the disclosure may be administered once or several times, also intermittently, for instance on a daily, weekly, monthly, or quarterly, or yearly basis for several days, weeks or months and in different dosages.

In another aspect, the immunogenic composition of the present disclosure is administered to an animal in need thereof at least two weeks of age. More preferably, the animal is between 2 weeks and 15 years of age, still more preferably between 3 weeks and 10 years of age. Of course, other ages and ranges are contemplated such as 2, 3, 4, 5, 6, 7, 8, 9, 10, 11, 12, 13, 14, 15, 16, 17, 18, 19, and 20 weeks or years or more. Alternatively, the immunogenic composition is administered at least 2 weeks prior to exposure to an Anaplasma bacteria.

In another aspect, the immunogenic composition of the present disclosure further includes at least one component selected from the group consisting of a veterinary-acceptable carrier, a pharmaceutical-acceptable carrier, an adjuvant, a preservative, a buffer, an antibiotic, cell culture supernatant, an immunomodulatory agent and any combination thereof.

In another aspect, the present disclosure provides a method of reducing the incidence of or severity of at least one clinical sign caused by an Anaplasma bacteria comprising the step of administering an immunogenic composition comprising a mutant Anaplasma bacteria having a disrupted or deleted gene therein and a component selected from the group consisting of a veterinary-acceptable carrier, a pharmaceutical-acceptable carrier, an adjuvant, a preservative, a buffer, an antibiotic, cell culture supernatant, an immunomodulatory agent, and any combination thereof. In some forms, the administering step is selected from the group consisting of intramuscularly, intranasally, intradermally, intratracheally, intravaginally, intravenously, intravascularly, intraarterially, intraperitoneally, orally, intrathecally, or by direct injection into target tissues. In some forms, the administration of the immunogenic composition can be termed a first administration and this first administration is followed by a second administration. In some forms, the second administration is at least 7 days after the first administration. In preferred forms, the reduction in incidence is in a group of animals that have received an administration of the immunogenic composition and at least 10, 20, 30, 40, 50, 60, 70, 80, 90, 91, 92, 93, 94, 95, 96, 97, 98, 99, or even 100% is in comparison to a group of animals that have not received an administration of the immunogenic composition of this disclosure. In preferred forms, the reduction in severity is assessed in a single animal that has received an administration of the immunogenic composition of this disclosure and is in comparison to an animal that has not received the immunogenic composition. In preferred forms, this reduction in severity is at least 10, 20, 30, 40, 50, 60, 70, 80, 90, 91, 92, 93, 94, 95, 96, 97, 98, 99, or even 100% when comparing an animal that has received the composition with an animal that has not received the immunogenic composition and that has been subsequently infected or challenged by an Anaplasma bacteria. In some forms, the reduction in severity in a group of animals that have received the immunogenic composition is at least 10, 20, 30, 40, 50, 60, 70, 80, 90, 91, 92, 93, 94, 95, 96, 97, 98, 99, or even 100% in comparison to a group of animals that have not received an administration of the immunogenic composition. In some forms, the component is an adjuvant selected from the group consisting of a saponin, cyclic GMP-AMP, montanide gel, or any combination thereof.

Those of skill in the art will understand that the immunogenic composition used herein may incorporate known injectable, physiologically acceptable sterile solutions for preparing a ready-to-use solution for parenteral injection or infusion, aqueous isotonic solutions, such as e.g. saline or corresponding plasma protein solutions, are readily available. In addition, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Stabilizers include albumin and alkali salts of ethylendiamintetracetic acid, among others.

In one aspect, the immunogenic composition may also comprise additional elements, antigens, pharmaceutical-acceptable carriers, veterinary-acceptable carriers, adjuvants, preservatives, stabilizers, or any combination thereof.

“Adjuvants” as used herein, can include aluminum hydroxide and aluminum phosphate, saponins, Quil A, cyclic GMP-AMP, montanide gel, QS-21 (Cambridge Biotech Inc., Cambridge MA), GPI-0100 (Galenica Pharmaceuticals, Inc., Birmingham, AL), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed. Stewart-Tull, D. E. S.). JohnWiley and Sons, NY, pp51-94 (1995) and Todd et al., Vaccine 15:564-570 (1997). For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book. Another preferred adjuvant is AddaVax (Invivogen, San Diego, CA, USA). A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. Further suitable adjuvants include, but are not limited to, the RIBI adjuvant system (Ribi Inc.), Block co-polymer (CytRx, Atlanta GA), SAF-M (Chiron, Emeryville CA), monophosphoryl lipid A, Avridine lipid-amine adjuvant, heat-labile enterotoxin from E. coli (recombinant or otherwise), cholera toxin, IMS 1314, or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

Additionally, the composition can include one or more pharmaceutical-acceptable or veterinary-acceptable carriers. As used herein, “a pharmaceutical-acceptable carrier” or “a veterinary-acceptable carrier” includes any and all solvents, dispersion media, coatings, stabilizing agents, diluents, preservatives, antibacterial and antifungal agents, isotonic agents, adsorption delaying agents, and the like.

Pharmaceutically acceptable vehicle is understood as designating a compound or a combination of compounds entering into a pharmaceutical composition or vaccine which does not provoke secondary reactions and which allows, for example, the facilitation of the administration of the active compound, an increase in its duration of life and/or its efficacy in the body, an increase in its solubility in solution or alternatively an improvement in its conservation. These pharmaceutically acceptable vehicles are well known and will be adapted by the person skilled in the art as a function of the nature and of the mode of administration of the chosen active compound.

For example, the immunogenic composition or vaccine according to the present disclosure may be administered one time or several times, spread out over time in an amount of about 0.1 to 1000 μg per kilogram weight of the animal or human, where values and ranges such as, but not limited to, 0.5 to 800 μg per kilogram weight of the animal or human, 1 to 1000 μg per kilogram weight of the animal or human, 1 to 500 μg per kilogram weight of the animal or human, 1 to 300 μg per kilogram weight of the animal or human, 1 to 200 μg per kilogram weight of the animal or human, 1 to 150 μg per kilogram weight of the animal or human, 1 to 125 μg per kilogram weight of the animal or human, 1 to 100 μg per kilogram weight of the animal or human, 5 μg per kilogram weight of the animal or human, 10 μg per kilogram weight of the animal or human, 15 μg per kilogram weight of the animal or human, 20 μg per kilogram weight of the animal or human, 25 μg per kilogram weight of the animal or human, 30 μg per kilogram weight of the animal or human, 35 μg per kilogram weight of the animal or human, 40 μg per kilogram weight of the animal or human, 45 μg per kilogram weight of the animal or human, 50 μg per kilogram weight of the animal or human, 55 μg per kilogram weight of the animal or human, 60 μg per kilogram weight of the animal or human, 65 μg per kilogram weight of the animal or human, 70 μg per kilogram weight of the animal or human, 75 μg per kilogram weight of the animal or human, 80 μg per kilogram weight of the animal or human, 85 μg per kilogram weight of the animal or human, 90 μg per kilogram weight of the animal or human, 95 μg per kilogram weight of the animal or human, 100 μg per kilogram weight of the animal or human, 125 μg per kilogram weight of the animal or human, 150 μg per kilogram weight of the animal or human, 200 μg per kilogram weight of the animal or human, 250 μg per kilogram weight of the animal or human, 300 μg per kilogram weight of the animal or human, 400 μg per kilogram weight of the animal or human, 500 μg per kilogram weight of the animal or human, 600 μg per kilogram weight of the animal or human, 700 μg per kilogram weight of the animal or human, 800 μg per kilogram weight of the animal or human, 900 μg per kilogram weight of the animal or human, and 1000 μg per kilogram weight of the animal or human are envisioned. In other preferred forms, the above amounts are also provided without reference to the weight of the animal or human.

“Sequence Identity” as it is known in the art refers to a relationship between two or more polypeptide (poly amino acid) sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or amino acid residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48:1073 (1988), the teachings of which are incorporated herein by reference. Preferred methods to determine the sequence identity are designed to give the largest match between the sequences tested. Methods to determine sequence identity are codified in publicly available computer programs, which determine sequence identity between given sequences. Examples of such programs include, but are not limited to, the GCG program package (Devereux, J., et al., Nucleic Acids Research, 12 (1): 387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, MD 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homolog sequence comprises at least a stretch of 50, even more preferably at least 100, even more preferably at least 250, and even more preferably at least 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly.

Any strain of A. marginale can be modified as disclosed herein and used as an antigenic component of the immunogenic composition of the disclosure. Additionally, the disruption or deletion of the phtcp gene is effective for all A. marginale strains.

### BRIEF DESCRIPTION OF THE DRAWINGS

FIG. 1 is a schematic representation of a plasmid map of AM581-KO-tuf-mCherry-Gent construct. Left and right homology arms for targeted allelic exchange are shown on the plasmid map. The segment containing the left and right homology arms separated by the E. chaffeensis tuf promoter segment to drive the expression of mCherry and gentamicin resistance cassette were amplified for use in allelic exchange homologous recombination;

FIG. 2A is a schematic representation of the genomic region selected for preparing the allelic exchange construct and construct design. Restriction enzyme sites used in defining the mutation development [EcoRV (E) and HindIII (H)] are presented with their genomic coordinates. The size of the inserted fragment (tuf-mCherry-Gent) were included. The PCR primers used in the analysis were identified as numbers 1 to 4;

FIG. 2B is a magnified photo of the AM581 gene deletion mutant in ISE6 tick cell culture expressing mCherry. The mCherry expression was detected by confocal microscopy using a 40× magnification lens;

FIG. 2C is a set of three photographs illustrating the use of PCR analysis to define the mutation. Three different PCRs were performed: in PCR I, primers targeting the genomic region upstream to the insertion and the mCherry coding sequence were used; PCR II primers targeted to the mCherry coding region and downstream to the inserted region; in PCRIII, primers were targeted to A. marginale sequence upstream and downstream to the inserted fragment. (L, 1 kb plus molecular weight DNA markers; W, wild-type A. marginale genomic DNA; M, phtcp mutant genomic DNA);

FIG. 2D is a Southern blot analysis of genomic DNAs (W, wild-type and M, mutant) digested with EcoRV (E) and HindIII (H). The DNA blot analysis was performed using an mCherry gene segment as the probe;

FIG. 3A is a graph illustrating PCV counts assessed in the infection control group of steers. Average PCV values from animals sampled during the study period were presented for the non-vaccinated animals during vaccination phage and following infection challenge. Days of adjuvant injections (control group) and infection challenge day are identified in the figures. At significance level α=0.05, One-way ANOVA with repeated measures showed significant differences for PCV (P=0.0128*) levels among the infection control, WCAV and MLAV groups for several days post-challenge. Additionally, Tukey's multiple comparisons test showed statistically significant differences in PCV (P=0.0056**) levels between infection control and MLAV groups, but there was no statistically significant difference between infection control and WCAV group animals.

FIG. 3B is a graph illustrating PCV counts in the WCAV group of steers. Average PCV values from animals sampled during the study period were presented for the WCAV-vaccinated animals during vaccination phage and following infection challenge. Days of vaccination injections and infection challenge day are identified in the figures. At significance level α=0.05, One-way ANOVA with repeated measures showed significant differences for PCV (P=0.0128*) levels among the infection control, WCAV and MLAV groups for several days post-challenge. Additionally, Tukey's multiple comparisons test showed statistically significant differences in PCV (P=0.0056**) levels between infection control and MLAV groups, but there was no statistically significant difference between infection control and WCAV group animals.

FIG. 3C is a graph illustrating PCV counts in the MLAV group of steers. Average PCV values from animals sampled during the study period were presented for the MLAV-vaccinated animals during vaccination phage and following infection challenge. Days of vaccination injections and infection challenge day are identified in the figures. At significance level α=0.05, One-way ANOVA with repeated measures showed significant differences for PCV (P=0.0128*) levels among the infection control, WCAV and MLAV groups for several days post-challenge. Additionally, Tukey's multiple comparisons test showed statistically significant differences in PCV (P=0.0056**) levels between infection control and MLAV groups, but there was no statistically significant difference between infection control and WCAV group animals.

FIG. 3D is a graph illustrating RBC counts in the infection control group of steers. Average RBC values from animals sampled during the study period were presented for the non-vaccinated animals during vaccination phage and following infection challenge. Days of adjuvant injections (control group) and infection challenge day (all three groups) are identified in the figures. At significance level α=0.05, One-way ANOVA with repeated measures showed significant differences for RBC (P=0.0375*) levels among the infection control, WCAV and MLAV groups for several days post-challenge. Additionally, Tukey's multiple comparisons test showed statistically significant differences in RBC P=0.0294*) levels between infection control and MLAV groups, but there was no statistically significant difference between infection control and WCAV group animals.

FIG. 3E is a graph illustrating RBC counts in the WCAV group of steers. Average RBC values from animals sampled during the study period were presented for the WCAV-vaccinated animals during vaccination phage and following infection challenge. Days of adjuvant injections (control group) and infection challenge day (all three groups) are identified in the figures. At significance level α=0.05, One-way ANOVA with repeated measures showed significant differences for RBC (P=0.0375*) levels among the infection control, WCAV and MLAV groups for several days post-challenge. Additionally, Tukey's multiple comparisons test showed statistically significant differences in RBC P=0.0294*) levels between infection control and MLAV groups, but there was no statistically significant difference between infection control and WCAV group animals.

FIG. 3F is a graph illustrating RBC counts in the MLAV group of steers. Average RBC values from animals sampled during the study period were presented for the MLAV-vaccinated animals during vaccination phage and following infection challenge. Days of adjuvant injections (control group) and infection challenge day (all three groups) are identified in the figures. At significance level α=0.05, One-way ANOVA with repeated measures showed significant differences for RBC (P=0.0375*) levels among the infection control, WCAV and MLAV groups for several days post-challenge. Additionally, Tukey's multiple comparisons test showed statistically significant differences in RBC P=0.0294*) levels between infection control and MLAV groups, but there was no statistically significant difference between infection control and WCAV group animals.

FIG. 4A is a photograph of thin blood smears from the infection control group assessed by light microscopy. Blood smear images were presented from one animal each for day 42 post infection challenge from all three experimental group animals. The images were collected following viewing with a 40× magnification. A. marginale inclusions are shown by black arrows (observed only in infection controls and WCAV animals; FIGS. 4A and 4B), but not in MLAV animals (FIG. 4C). The A. marginale inclusions (identified with arrowhead lines) and anisocytosis are evident in both non-vaccinated infection controls WCAV vaccinees, but not in MLAV vaccinees. The length bars on the bottom left corner of each panel represent 50 microns.

FIG. 4B is a photograph of thin blood smears from the WCAV-vaccinated group assessed by light microscopy. Blood smear images were presented from one animal each for day 42 post infection challenge from all three experimental group animals. The images were collected following viewing with a 40× magnification. A. marginale inclusions are shown by black arrows (observed only in infection controls and WCAV animals; FIGS. 4A and 4B), but not in MLAV animals (FIG. 4C). The A. marginale inclusions (identified with arrowhead lines) and anisocytosis are evident in both non-vaccinated infection controls WCAV vaccinees, but not in MLAV vaccinees. The length bars on the bottom left corner of each panel represent 50 microns.

FIG. 4C is a photograph of thin blood smears from the MLAV-vaccinated group assessed by light microscopy. Blood smear images were presented from one animal each for day 42 post infection challenge from all three experimental group animals. The images were collected following viewing with a 40× magnification. A. marginale inclusions are shown by black arrows (observed only in infection controls and WCAV animals; FIGS. 4A and 4B), but not in MLAV animals (FIG. 4C). The A. marginale inclusions (identified with arrowhead lines) and anisocytosis are evident in both non-vaccinated infection controls WCAV vaccinees, but not in MLAV vaccinees. The length bars on the bottom left corner of each panel represent 50 microns.

FIG. 5A is a graph of percent infected erythrocytes as measured by light microscopy for the infection controls. Average percent infected erythrocytes following enumeration in 20 randomly chosen fields under 40× magnification and presented for all groups throughout the study period. Days of vaccinations and infection challenges were as in FIGS. 3A-3F. At significance level α=0.05, One-way ANOVA with repeated measures showed that percent infected erythrocytes (bacteremia) were significantly different between the infection control, WCAV and MLAV groups (P=0.0058**), for several days post-challenge. Additionally, Tukey's multiple comparisons test showed statistically significant differences between infection control and MLAV (FIG. 5C) (P=0.0334*), as well as between WCAV (FIG. 5B) and MLAV (FIG. 5C) (P=0.0117*) groups. No statistically significant difference between infection control and WCAV (FIG. 5B) group animals was observed.

FIG. 5B is a graph of percent infected erythrocytes as measured by light microscopy for the WCAV-vaccinated group. Average percent infected erythrocytes following enumeration in 20 randomly chosen fields under 40× magnification and presented for all groups throughout the study period. Days of vaccinations and infection challenges were as in FIGS. 3A-3F. At significance level α=0.05, One-way ANOVA with repeated measures showed that percent infected erythrocytes (bacteremia) were significantly different between the infection control, WCAV and MLAV groups (P=0.0058**), for several days post-challenge. Additionally, Tukey's multiple comparisons test showed statistically significant differences between infection control (FIG. 5A) and MLAV (P=0.0334*), as well as between WCAV and MLAV (FIG. 5C) (P=0.0117*) groups. No statistically significant difference between infection control and WCAV group animals was observed.

FIG. 5C is a graph of percent infected erythrocytes as measured by light microscopy for the MLAV-vaccinated group. Average percent infected erythrocytes following enumeration in 20 randomly chosen fields under 40× magnification and presented for all groups throughout the study period. Days of vaccinations and infection challenges were as in FIGS. 3A-3F. At significance level α=0.05, One-way ANOVA with repeated measures showed that percent infected erythrocytes (bacteremia) were significantly different between the infection control, WCAV (FIG. 5A) and MLAV (FIG. 5B) groups (P=0.0058**), for several days post-challenge. Additionally, Tukey's multiple comparisons test showed statistically significant differences between infection control (FIG. 5A) and MLAV (P=0.0334*), as well as between WCAV (FIG. 5B) and MLAV (P=0.0117*) groups. No statistically significant difference between infection control (FIG. 5A) and WCAV group animals was observed.

FIG. 6A is a graph illustrating bacterial numbers in blood assessed by real-time qPCR for the infection control group. The 16S rDNA TaqMan probe-based qPCR assays were performed for samples collected from all three groups animals over the study period. The MLAV group animal samples were also assessed by mCherry qPCR assay (FIG. 6D). At significance level α=0.05, One-way ANOVA with repeated measures showed that 16S rDNA copy numbers of wild-type A. marginale bacteria were significantly different between the infection control, WCAV (FIG. 6B) and MLAV (FIG. 6C) groups (P=0.0375*), for several days post-challenge. Additionally, Tukey's multiple comparisons test showed a statistically significant difference between infection control and MLAV (FIG. 6C) groups (P=0.0294*). No statistically significant difference was observed between infection control and WCAV (FIG. 6B).

FIG. 6B is a graph illustrating bacterial numbers in blood assessed by real-time qPCR for the WCAV-vaccinated group. The 16S rDNA TaqMan probe-based qPCR assays were performed for samples collected from all three groups animals over the study period. The MLAV group animal samples were also assessed by mCherry qPCR assay (FIG. 6D). At significance level α=0.05, One-way ANOVA with repeated measures showed that 16S rDNA copy numbers of wild-type A. marginale bacteria were significantly different between the infection control (FIG. 6A), WCAV and MLAV (FIG. 6C) groups (P=0.0375*), for several days post-challenge. Additionally, Tukey's multiple comparisons test showed a statistically significant difference between infection control (FIG. 6A) and MLAV (FIG. 6C) groups (P=0.0294*). No statistically significant difference was observed between infection control (FIG. 6A) and WCAV.

FIG. 6C is a graph illustrating bacterial numbers in blood assessed by real-time qPCR for the MLAV-vaccinated group. The 16S rDNA TaqMan probe-based qPCR assays were performed for samples collected from all three groups animals over the study period. The MLAV group animal samples were also assessed by mCherry qPCR assay (FIG. 6D). At significance level α=0.05, One-way ANOVA with repeated measures showed that 16S rDNA copy numbers of wild-type A. marginale bacteria were significantly different between the infection control, WCAV (FIG. 6B) and MLAV (FIG. 6C) groups (P=0.0375*), for several days post-challenge. Additionally, Tukey's multiple comparisons test showed a statistically significant difference between infection control and MLAV (FIG. 6C) groups (P=0.0294*). No statistically significant difference was observed between infection control and WCAV (FIG. 6B).

FIG. 6D is a graph illustrating bacterial numbers in blood assessed by real-time qPCR for the MLAV-vaccinated groups. The MLAV group animal samples were assessed by mCherry qPCR assay. At significance level α=0.05, One-way ANOVA with repeated measures showed that 16S rDNA copy numbers of wild-type A. marginale bacteria were significantly different between the infection control (FIG. 6A), WCAV (FIG. 6B) and MLAV groups (P=0.0375*), for several days post-challenge. Additionally, Tukey's multiple comparisons test showed a statistically significant difference between infection control (FIG. 6A) and MLAV groups (P=0.0294*). No statistically significant difference was observed between infection control and WCAV (FIG. 6B).

FIG. 7 is a series of photographs illustrating the presence of wild-type and mutant A. marginale in ticks assessed following blood feeding on MLAV group animals and their subsequent molting. Genomic DNAs from 20 ticks fed on each animal (10 males and 10 females) from steers 4506 and HH5 were tested for the presence of wild-type and phtcp mutant A. marginale by conventional PCR targeting to amplify the entire insertion-specific region; anticipated product size for wild-type and mutant are 2.4 kb and 3.4 kb, respectively. As a total of 60 randomly selected ticks (30 males and 30 females) fed on animal #DP324 were negative for both wild-type and mutant A. marginale by qPCR, 10 randomly selected ticks from this animal were also tested by conventional PCR. L, 1 kb plus molecular weight markers; M, A. marginale mutant; W, Wild-type strain,-refers to no template containing negative control. The numbers with the letters M or F are to indicate the tick identification numbers and to indicate their sex; M, male and F, female.

FIG. 8A is a set of photographs illustrating post infection challenge histopathology sections of bone marrow at 40× magnification from WCAV vaccinees (animals #s 4491, 4502 and 4505);

FIG. 8B is a set of photographs illustrating post infection challenge histopathology sections of bone marrow at 40× magnification from MLAV vaccinees (animal #s DP324, 4506 and HH5).

FIG. 9A is a graph illustrating A. marginale-specific IgG response in the infection control group of cattle. Antigen-specific IgG antibodies were measured by ELISA in plasma samples collected from day zero (prior to infection) and multiple time points post vaccination and post challenge. Purified A. marginale whole cell antigens recovered from ISE6 cell cultures were used to coat the ELISA plates. Average absorbance values of plasma collected from steers within each group were plotted against plasma collection days. At, significance level α=0.05, One-way ANOVA with repeated measures showed that IgG levels were significantly different between the infection control, WCAV (FIG. 9B) and MLAV (FIG. 9C) groups (P<0.0001****), for several days post-challenge. Additionally, Tukey's multiple comparisons test showed significant differences between infection control and WCAV (FIG. 9B) groups (P=0.0003***), infection control and MLAV (FIG. 9C) groups (P=0.0024**), and WCAV (FIG. 9B) and MLAV (FIG. 9C) groups (P=0.0041**).

FIG. 9B is a graph illustrating A. marginale-specific IgG response in the WCAV-vaccinated group of cattle. Antigen-specific IgG antibodies were measured by ELISA in plasma samples collected from day zero (prior to infection) and multiple time points post vaccination and post challenge. Purified A. marginale whole cell antigens recovered from ISE6 cell cultures were used to coat the ELISA plates. Average absorbance values of plasma collected from steers within each group were plotted against plasma collection days. At, significance level α=0.05, One-way ANOVA with repeated measures showed that IgG levels were significantly different between the infection control (FIG. 9A), WCAV and MLAV (FIG. 9C) groups (P<0.0001****), for several days post-challenge. Additionally, Tukey's multiple comparisons test showed significant differences between infection control (FIG. 9A) and WCAV groups (P=0.0003***), infection control (FIG. 9A) and MLAV (FIG. 9C) groups (P=0.0024**), and WCAV and MLAV (FIG. 9C) groups (P=0.0041**).,

FIG. 9C is a graph illustrating A. marginale-specific IgG response in the MLAV-vaccinated cattle. Antigen-specific IgG antibodies were measured by ELISA in plasma samples collected from day zero (prior to infection) and multiple time points post vaccination and post challenge. Purified A. marginale whole cell antigens recovered from ISE6 cell cultures were used to coat the ELISA plates. Average absorbance values of plasma collected from steers within each group were plotted against plasma collection days. At, significance level α=0.05, One-way ANOVA with repeated measures showed that IgG levels were significantly different between the infection control (FIG. 9A), WCAV (FIG. 9B) and MLAV groups (P<0.0001****), for several days post-challenge. Additionally, Tukey's multiple comparisons test showed significant differences between infection control (FIG. 9A) and WCAV (FIG. 9B) groups (P=0.0003***), infection control (FIG. 9A) and MLAV groups (P=0.0024**), and WCAV (FIG. 9B) and MLAV groups (P=0.0041**).

FIG. 10 is a graph assessing PCV in a nonvaccinated group and a vaccinated group of steers following vaccination and A. marginale tick transmission. Average PCV values from three animals sampled during the study period were presented.

FIG. 11 is a graph of bacteremia assessed by qPCR in a nonvaccinated group and a vaccinated group of steers following vaccination and A. marginale tick transmission. Average bacterial numbers from animals sampled during the study period were presented.

FIG. 12 is a series of photograph of significant blood cells having healthy morphology in vaccinated (panels A-C) and abnormalities in nonvaccinated (panels D-F) steers was observed and detailed below in Table 3. Panel A is normal red blood cells, panel B is normal lymphocytes, panel C is normal neutrophils, panel D is red blood cells with reticulocyte (arrow), anisocytosis (varying sizes), and morula (arrowhead), panel E is activated lymphocytes, and panel F is a band cell.

FIG. 13 is a graph illustrating A. marginale-specific IgG response in cattle following MLAV vaccination and/or tick transmitted infection challenge. Antigen-specific IgG antibodies were measured by ELISA in plasma samples collected from day zero (prior to infection) and multiple time points post vaccination and post challenge.

### DETAILED DESCRIPTION OF THE PRESENT DISCLOSURE

The following detailed description and examples set forth preferred materials and procedures used in accordance with the present disclosure. It is to be understood, however, that this description and these examples are provided by way of illustration only, and nothing therein shall be deemed to be a limitation upon the overall scope of the present disclosure.

### Example 1

### Materials and Methods

In vitro cultivation of A. marginale: Both the wild-type and the phtcp mutant A. marginale St. Maries strain were propagated in Ixodes scapularis cell line culture (ISE6) at 34° C. in the absence of CO2 as described earlier (Felsheim R F, et al. Transformation of Anaplasma marginale. Vet Parasitol. 2010; 167:167-174) (the teachings and content of which are hereby incorporated by reference herein), except that the media for culturing the mutant included gentamicin at a final concentration of 60 μg/ml.

Generation of AM581 deletion construct: The homology arms of 1.1 kb each from both 5′ and 3′ to the phtcp gene (gene tag #AM581) of A. marginale St. Maries strain (GenBank #: CP000030) were amplified with the PCR primer sets listed in Table 1 and using the bacterial genomic DNA as the template. The A. marginale PCR products and a previously generated plasmid construct containing the contiguous E. chaffeensis tuf promoter and ORFs of mCherry and the gentamicin resistance genes (tuf-mCherry-Gent) (Wang Y, et al., A genetic system for targeted mutations to disrupt and restore genes in the obligate bacterium, Ehrlichia chaffeensis. Sci Rep. 2017; 7:1-13) (the teachings and contents of which are hereby incorporated by reference herein), were cloned into the pGGA plasmid vector (New England Biolabs, Ipswich, USA). The Golden Gate Assembly kit was used to assemble the fragments into the pGGA plasmid in the following order: 5′ A. marginale homology arm (1.1 kb), tuf-mCherry-Gent segment (1.6 kb), and 3′ A. marginale homology arm (1.1 kb). The final assembled recombinant plasmid is referred to as ‘AM581-KO-tuf-mCherry-Gent’. Standard molecular cloning protocols were followed to recover the recombinant plasmid transformed into the DH5x strain of E. coli. The integrity of the plasmid DNA, purified from the transformed E. coli, was verified by Sanger's DNA sequencing analysis using the commercially available T7 and SP6 promoter primers (Integrated DNA Technologies, Coralville, IA, USA) annealing to the pGGA plasmid backbone. The recombinant plasmid was used as the template in a PCR to amplify the fragment containing the 5′ A. marginale homology arm, the tuf-mCherry-Gent segment, and the 3′ A. marginale homology arm (primers listed in Table 1). The PCR products were then purified as reported previously (Wang et al., 2017).

TABLE 1
List of oligonucleotides used in this study

| SEQ ID | | | Target/ |
|---|---|---|---|
| NO. | Name | Sequence (5′-3′) | Purpose |
| SEQ ID | RG2073 | ATCGGTGGTCTCCGGAGTT | AM581 Left |
| NO. 1 | | TCGCTATACAGAGCAGAA | Homology |
| SEQ ID | RG2056 | ATCGGTGGTCTCCAACTAA | Arm |
| NO. 2 | | ACCACAGTGAAATTTTTAA | |
| | | GA | |
| SEQ ID | RG2059 | ATCGGTGGTCTCGTTGTGA | AM581 |
| NO. 3 | | ACATTGCAGACCTG | Right |
| SEQ ID | RG2074 | ATCGGTGGTCTCGATGGAT | Homology |
| NO. 4 | | ATCGGCCCTTGCTGTC | Arm |
| SEQ ID | RG2057 | ATCGGTGGTCTCCAGTTTA | Tuf- |
| NO. 5 | | TGTTGCTGTACTTGGATC | mCherry- |
| SEQ ID | RG2058 | ATCGGTGGTCTCCACAAAA | Gentamycin |
| NO. 6 | | ATGTGACTATTAATTTTGA | |
| | | CTTTT | |
| SEQ ID | RG94 | AAGCAAATGCTTTAGGTGC | PCR I |
| NO. 7 | | AT* | |
| SEQ ID | RG2083 | TCGGAGGTAGCGTGTCCTT | |
| NO. 8 | | A | |
| SEQ ID | RG97 | TCCGCAGGATGTTTCACAT | PCR II |
| NO. 9 | | A* | |
| SEQ ID | RG2084 | GCATGGGCGTGGGTTTTTA | |
| NO. 10 | | G | |
| SEQ ID | RG2083 | TCGGAGGTAGCGTGTCCTT | PCR III |
| NO. 11 | | A | |
| SEQ ID | RG2084 | GCATGGGCGTGGGTTTTTA | |
| NO. 12 | | G | |
| SEQ ID | RG2151 | CTCAGAACGAACGCTGG | 16S rDNA |
| NO. 13 | | | qPCR |
| SEQ ID | RG2152 | CATTTCTAGTGGCTATCCC | |
| NO. 14 | | | |
| SEQ ID | RG2152P | FAM/CGCAGCTTG/ZEN/C | 16S rDNA |
| NO. 15 | | TGCGTGTATGGT/IABkFQ | qPCR probe |
| SEQ ID | RG2177 | CGCGTGGGATATTCTTTC | mCherry |
| NO. 16 | | | qPCR |
| SEQ ID | RG2178 | CCGGGAAAGACAGTTTAAG | |
| NO. 17 | | | |
| SEQ ID | RG2179 | FAM/AGCCTATGT/ZEN/G | mCherry |
| NO. 18 | | AAACATCCTGCGGA/ | qPCR probe |
| | | IABkFQ | |
| SEQ ID | RG2161 | ACAATCTCTCGGCAGGCAA | AM581 |
| NO. 19 | | A | (phtcp) |
| SEQ ID | RG2162 | CGGTCATGGAATCTCGCCT | gene |
| NO. 20 | | T | (internal) |

*Obtained from Wang et al. (2017).

Generation, clonal purification, verification, and propagation of A. marginale phtcp mutant: Approximately 20 μg of the above purified amplicon from the AM581-KO-tuf-mCherry-Gent plasmid were electroporated into ISE6 tick cell culture-derived A. marginale St. Maries organisms (˜3×108), by following our previously described method (Wang et al., 2017). The electroporated bacteria were transferred to a cell suspension containing approximately 1×106 uninfected ISE6 tick cells and propagated at 34° C. in a T25 culture flask containing tick cell media for 24 h and then supplemented with 60 μg/ml final concentration of gentamicin. The cultures were maintained in the media with media changes once a week for the first three weeks and twice a week thereafter. The presence of mutant A. marginale expressing mCherry in cultures was monitored by fluorescence microscopy, while also maintaining several weeks in the presence of gentamicin to clear all wild-type bacteria.

To confirm the clonal purity of the mutant, three different PCR assays were performed using genomic DNA recovered from the mutant cultures; 1) forward primer specific to the inserted gentamicin gene segment and reverse primer targeted to the upstream genomic region 2) forward primer targeted to the downstream genomic region and reverse primer specific to the inserted mCherry gene segment, and 3) and forward and reverse primers targeted to the genomic regions upstream and downstream to the gene deletion-insertion mutation region (all primers are listed in Table 1). The PCR assays were performed in 25 μl reactions in 1x Q5 reaction buffer containing 2 mM MgCl2, 0.5 mM of each dNTP, 0.2 μM of each forward and reverse primers, 1 unit of Q5 Taq polymerase (New England Biolabs, Ipswich, MA, USA), and genomic DNA from wild-type or mutant organisms as the templates. The PCR cycling conditions for the first two PCRs were 98° C. for 30 s, followed by 35 cycles of 98° C. for 10 s, 65° C. for 30 s, and 72° C. for 2 min 30 s, then 72° C. for 3 mins and a final hold at 10° C. For the third PCR assay, the annealing temperature was changed to 70° C. The PCR products were resolved on a 1.5% agarose gel containing ethidium bromide and visualized using a UV transilluminator. Clonal purity of the mutant was further assessed by Southern blot analysis using the mutant culture-derived genomic DNA digested with HindIII or EcoRV restriction enzymes and genomic DNA from wild-type A. marginale was similarly digested and used to serve as the control. The insertion-specific mCherry gene segment-specific DNA probe was used for detecting approximately 4.3 kb and 3.7 kb DNA fragments, respectively, only in genomic DNA recovered from the mutant cultures.

A. marginale WCAV preparation: Purified wild-type A. marginale St. Maries strain organisms recovered from ISE6 cell cultures were heat inactivated at 60° C. for 30 min (referred as the whole cell inactivated antigen; WCA). The protein concentration of the WCA was estimated by the BCA protein estimation method (ThermoFisher Scientific, Carlsbad, CA, USA). Approximately 200 μg of WCA per 1 ml 1×PBS was mixed with an equal volume of oil-in-water suspension adjuvant, AddaVax (Invivogen, San Diego, CA, USA), for use as subcutaneously administered vaccine (WCAV).

Cattle infection and vaccine studies: All experiments with cattle were performed in accordance with the Public Health Service (PHS) Policy on the Humane Care and Use of Laboratory Animals, the U.S. Department of Agriculture's (USDA) Animal Welfare Act & Regulations, and with the prior approval of the Kansas State University's Institutional Animal Care and Use Committee (IACUC) (protocol #4362). At the conclusion of the study, all animals were euthanized according to the institutional IACUC recommendations, which are consistent with the recommendations of the Panel on Euthanasia of the American Veterinary Medical Association. Ten Holstein steers, approximately 18 months old, were obtained from an area in North Dakota reported to be free of bovine anaplasmosis (animal numbers are listed in Table 2). To confirm no prior exposure, serum and whole blood from each animal were screened by an MSP5-based cELISA (Anaplasma Antibody Test Kit, cELISA v2; VMRD, Pullman, WA, USA) and A. marginale 16S rDNA qPCR [51], respectively. The steers were housed at a vector-free animal facility at Kansas State University with food and water provided ad libitum. Steers could interact and socialize within their respective group animals. Animals were individually housed when tick 5 studies were performed. Adequate space was also given to allow regular exercise/activity.

TABLE 2
TaqMan qPCR data (Ct values) for ticks fed on steers.
A. Infection control group

| | 4493 | | 4496 | | HH6 | |
|---|---|---|---|---|---|---|
| Animal ID | M | F | M | F | M | F |
| | 21.5 | 19.9 | 15.5 | 16.8 | 16.4 | 14.5 |
| | 26.7 | 21.0 | 16.1 | 15.7 | 15.5 | 15.1 |
| | 21.5 | 20.1 | 18.0 | 13.9 | 16.3 | 21.5 |
| | 16.6 | 19.5 | 18.1 | 19.0 | 20.1 | 19.5 |
| | -ve | 21.2 | 14.8 | 18.7 | 18.3 | 17.5 |
| | 17.7 | 18.8 | 20.3 | 16.0 | 20.3 | 18.8 |
| | 23.6 | 18.2 | 17.3 | 16.6 | 16.8 | 17.7 |
| | 21.8 | 17.8 | 17.4 | 15.4 | 13.7 | 17.0 |
| | 18.6 | 21.0 | 17.5 | 16.6 | 18.4 | 13.7 |
| | 18.3 | 17.7 | 16.3 | 19.6 | 17.7 | 17.2 |

B. WCAV group

| | 4491 | | 4502 | | 4505 | |
|---|---|---|---|---|---|---|
| Animal ID | M | F | M | F | M | F |
| | 22.9 | 21.2 | 34.8 | 22.4 | 22.8 | 23.6 |
| | 22.5 | 20.9 | 20.9 | 21.2 | 20.3 | 23.8 |
| | 24.7 | 21.2 | 22.6 | 20.2 | 21.4 | 22.9 |
| | 22.3 | 25.9 | 23.6 | 21.9 | 25.6 | 22.7 |
| | 20.5 | 24.9 | 21.3 | 23.3 | 23.7 | 21.8 |
| | 22.6 | 22.3 | 23.8 | 22.7 | 21.1 | 22.1 |
| | 22.2 | 22.8 | 21.7 | 20.1 | 24.0 | 20.8 |
| | 21.5 | 23.1 | 20.5 | 23.7 | 23.4 | 23.1 |
| | 25.4 | 20.5 | 26.5 | 21.9 | 21.0 | 21.9 |
| | 23.5 | 25.1 | 24.5 | 24.0 | 21.1 | -ve |

B. MLAV group

| | 16S rRNA | | mCherry | |
|---|---|---|---|---|
| Animal ID | M | F | M | F |
| HH5 | 26.7 | 23.1 | 27.8 | 25.3 |
| | 28.0 | -ve | 29.0 | 36.8 |
| | 23.4 | 23.2 | 24.8 | 24.1 |
| | 24.2 | 30.6 | 26.0 | 32.1 |
| | 27.3 | 26.5 | 28.0 | 28.4 |
| | 28.2 | 27.6 | 29.4 | 28.9 |
| | 31.0 | 25.5 | 32.3 | 26.5 |
| | 27.8 | 32.8 | 29.0 | 33.2 |
| | 32.5 | 22.7 | 34.6 | 26.0 |
| | 25.2 | 21.6 | 26.3 | 23.4 |
| 4506 | 23.2 | 36.2 | 24.1 | 33.8 |
| | 23.0 | 29.1 | 24.8 | 30.1 |
| | -ve | 26.8 | 37.9 | 28.0 |
| | 26.6 | 28.4 | 27.9 | 33.2 |
| | 30.1 | 31.3 | 31.0 | 35.3 |
| | 33.1 | -ve | 36.0 | 34.2 |
| | 24.7 | 27.3 | 29.0 | 33.4 |
| | 23.0 | 28.5 | 24.8 | 29.8 |
| | 24.5 | 28.9 | 26.0 | 30.3 |
| | 28.7 | -ve | 26.0 | 34.1 |
| DP324 | -ve | -ve | -ve | -ve |
| | -ve | -ve | -ve | -ve |
| | -ve | -ve | -ve | -ve |
| | -ve | -ve | -ve | -ve |
| | -ve | -ve | -ve | -ve |
| | -ve | -ve | -ve | -ve |
| | -ve | -ve | -ve | -ve |
| | -ve | -ve | -ve | -ve |
| | -ve | -ve | -ve | -ve |
| | -ve | -ve | -ve | -ve |

Infection experiments in steers: Infection experiments were performed using either the in vitro cultured mutant organisms or with virulent A. marginale St. Maries wild-type strain blood stabilates. For mutant A. marginale infection experiments, steers received ˜3×108 ISE6 tick cell culture-derived mutant organisms resuspended in 2 ml of 1×PBS. The infection challenges with virulent St. Maries strain were performed IV using 2 ml each of blood stabilate (originating from the same batch) as per the previously described protocol (Hammac G K, et al., Protective immunity induced by immunization with a live, cultured Anaplasma marginale strain. Vaccine. 2013; 31:3617-22) (the teachings and content of which are hereby incorporated by reference herein). The MLAV vaccinees were challenged with the virulent strain on day 28, while the WCAV group animals were challenged on day 35. Non-vaccinated infection control group steers received AddaVax adjuvant diluted in 1×PBS (1:1) during the WCAV vaccination days. Prior to infection, blood stabilates were mixed with 5 ml freshly collected homologous blood plasma. Animals in MLAV, WCAV, and non-vaccinated groups received the same batch of inoculum.

Animal monitoring, CBC, and assessment of systemic A. marginale: All cattle used in the current study were monitored daily for health and behavioral changes and twice weekly for body temperature or when an animal was clinically ill. Veterinary care for the animals was overseen by a Kansas State University veterinarian. Throughout the study, 20 ml of blood was collected in EDTA tubes each week from all animals for plasma analysis. About 2 ml of blood was similarly collected twice per week for CBC analysis, performed on a VetScan HM5 Hematology Analyzer v2.3 (Zoetis, Union City, CA, USA). A small fraction of blood also in EDTA tubes was collected every other day for preparation and light microscopic analysis of blood smears to monitor for erythrocyte A. marginale inclusions. Blood sampled from all animals were also assessed once per week for the presence of A. marginale by 16S rDNA PCR analysis. All blood samples were processed either immediately or stored at 4° C. for a maximum of 24 h prior to performing the described analyses. DNeasy Blood and Tissue DNA isolation kit (Qiagen, Germantown, MD, USA) was used to extract total genomic DNA from a 100 μl aliquot of the collection whole blood samples. Extracted genomic DNA from each sample was eluted in 150 μl of elution buffer. To assess A. marginale infection status, TaqMan probe-based qPCR assays were performed targeting the 16S rDNA. Animals receiving the mutant A. marginale strain were also tested for the mutant-specific qPCR assay targeting the mCherry gene. The assay was standardized using the primers and TaqMan probes listed in Table 1. The qPCR assays were performed in 25 μl reactions with final concentrations of 1×reaction buffer containing 0.4 mM of each dNTP, 2.4 mM MgSO4, 0.1 μM concentration of both forward and reverse primers and the TaqMan probes, 1 unit of Platinum Taq polymerase (ThermoFisher Scientific, Carlsbad, CA, USA), and by including 2 μl each of genomic DNA as a template. Genomic DNA extracted from the wild-type A. marginale St. Maries strain was included as the positive control for the 16S rDNA assays, while DNA from the mutant A. marginale was used as the positive control for assays targeting the mCherry gene. Negative controls included all reactants and PCR-grade water in place of DNA template. The qPCR cycling conditions for the assays were 95° C. for 3 mins, followed by 45 cycles of 94° C. for 15 s, 50° C. for 30 s and 60° C. for 1 min (signal acquisition stage). Serial dilutions of the 16S rDNA gene- and mCherry gene-containing plasmids were used in the assays to define the copy numbers of molecules in the respective test samples. The Ct values obtained by fluorescence signal detection of the serially diluted plasmid controls ranging from 109 to 101 copies were used for generating standard curves and all assays were performed in triplicate.

Xenodiagnosis of A. marginale by Dermacentor variabilis: Approximately 250 D. variabilis nymphal stage ticks were placed on all animals on day 19 post A. marginale wild-type infection challenge. Ticks were allowed to feed to repletion (˜10 days). Fed ticks were carefully collected from the tick attachment cells and transferred to a humidified incubator with 14 h day light and 10 h darkness for molting to the adult stage, which took approximately 30 days. Genomic DNAs from molted ticks (equal numbers of males and females) fed on each animal were initially isolated and subjected to qPCR targeting to A. marginale 16S rDNA. The mCherry gene qPCR assays were also performed on DNAs recovered from ticks fed on the MLAV animals. Genomic DNA extractions were performed using a Qiagen DNeasy Blood and Tissue Kit (Qiagen, Germantown, MD, USA). The purified genomic DNA from each tick was recovered in 150 μl each of the elution buffer and stored at −20° C. until use. A conventional PCR assay was also performed on tick DNAs using the forward and reverse primers targeted to the genomic regions upstream and downstream to the homology arm segments used in the mutagenesis experiment (primers listed in Table 1)

Assessment for the presence of A. marginale-specific IgG production in steers by ELISA:

Ninety-six well ELISA plates were coated with 10 μg/ml of host-cell free A. marginale total proteins prepared from the ISE6 tick cell-cultured organisms by incubating overnight at 4° C. The wells were blocked with the blocking buffer (1×PBS containing 1% BSA) and incubated at 37° C. for 1 h. Plasma samples from the animals were diluted 1:200 in the blocking buffer, added to wells and incubated at 37° C. for 1 h. The plates were then washed three times with wash buffer (1×PBS containing 0.05% Tween 20). Finally, the HRP conjugated anti-bovine IgG (Invitrogen, Frederick, MD, USA) at 1:2,000 dilution was added to the wells and incubated at 37° C. for 1 h. The ELISA plates were washed three times with the wash buffer and then TMB substrate (EMD Millipore Corporation, Temecula, CA, USA) was added. After observing color development in the wells, the reactions were stopped by adding 0.1 M phosphoric acid solution (stop solution) and the absorbance at 450 nm was measured using an ELISA reader (Biotek Instruments, Winooski, VT, USA). All assays were performed in triplicate and the mean absorbance values and standard deviation were calculated.

Statistical analysis: One-way ANOVA with repeated measures and Tukey's multiple comparisons tests were performed using GraphPad Software (La Jolla, CA, USA) at significance level, α=0.05, to assess the differences in A. marginale numbers in blood, PCV, RBC and IgG levels between the three groups at each time point following challenge.

### Results:

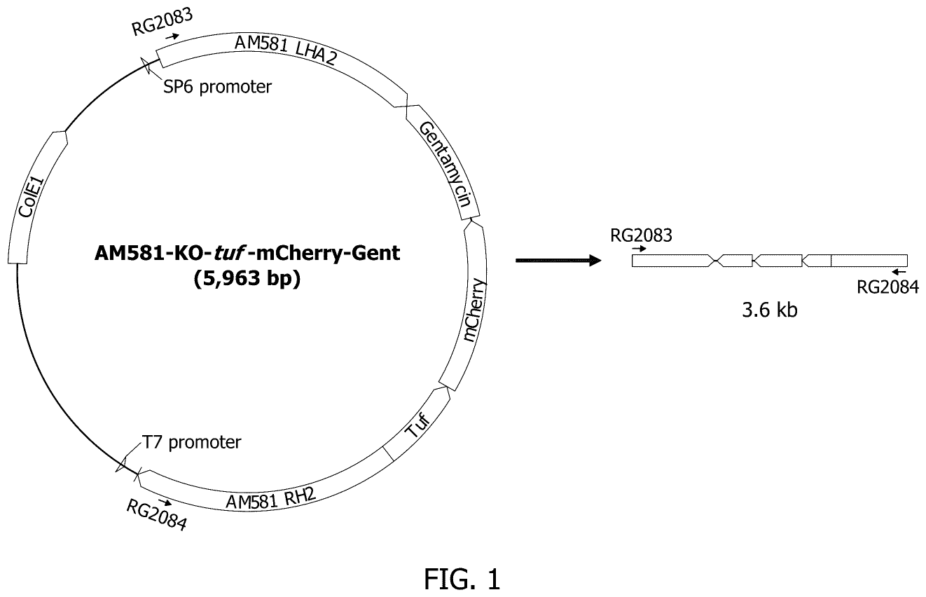

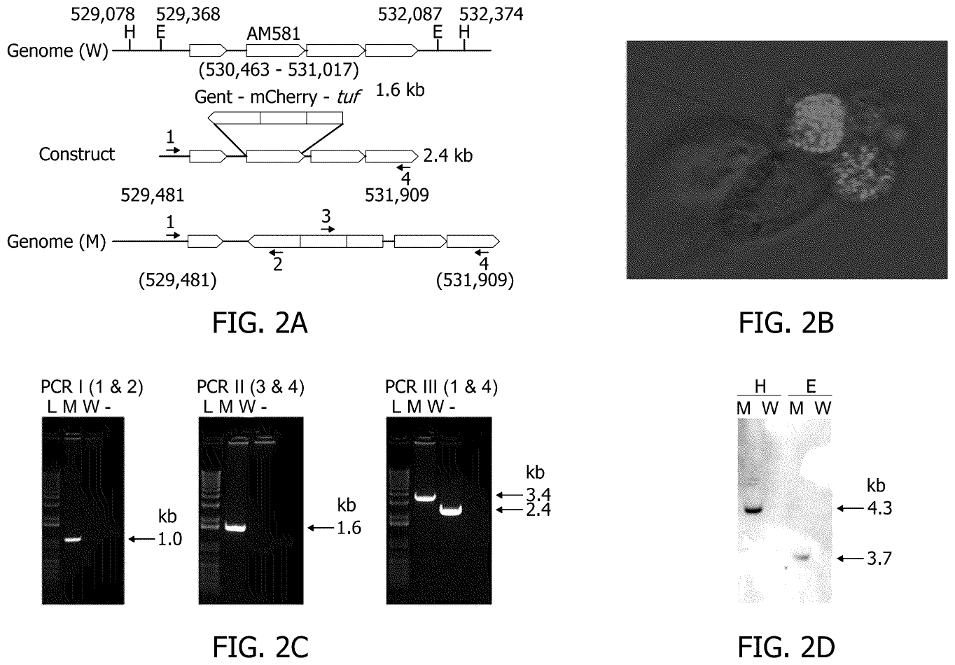

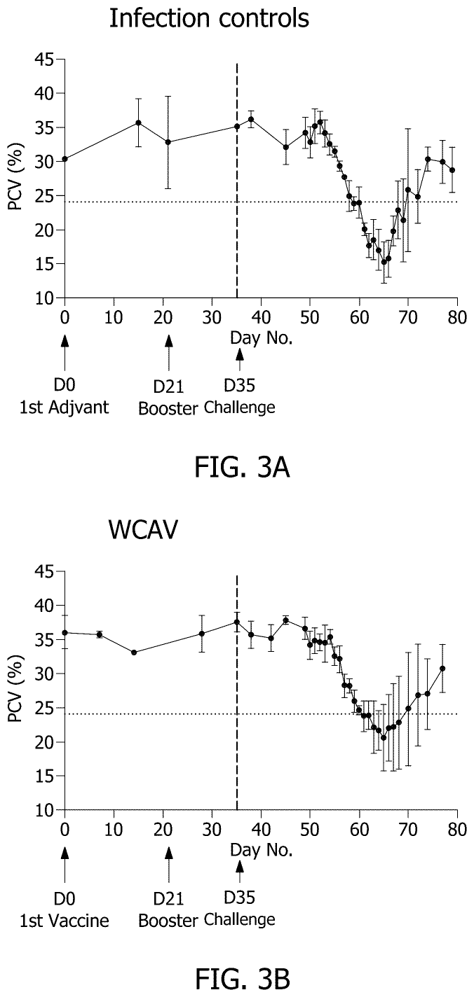

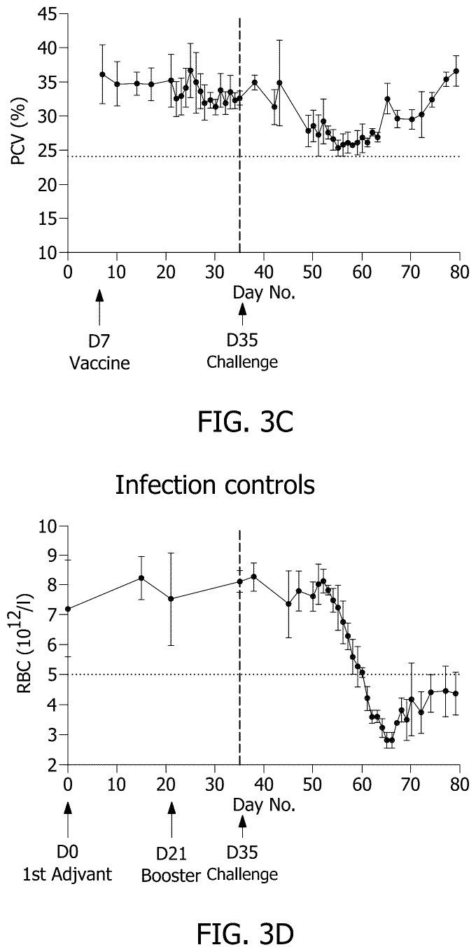

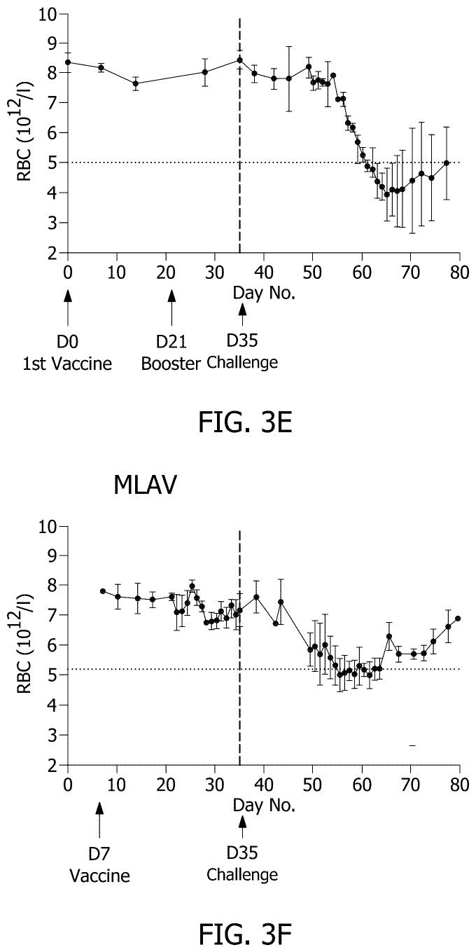

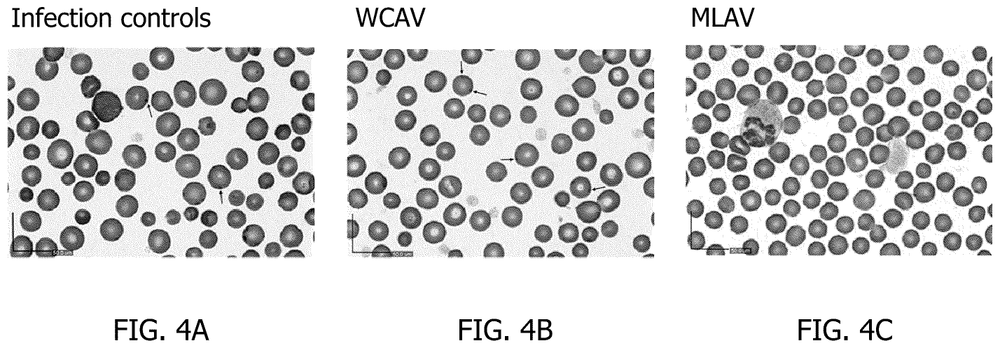

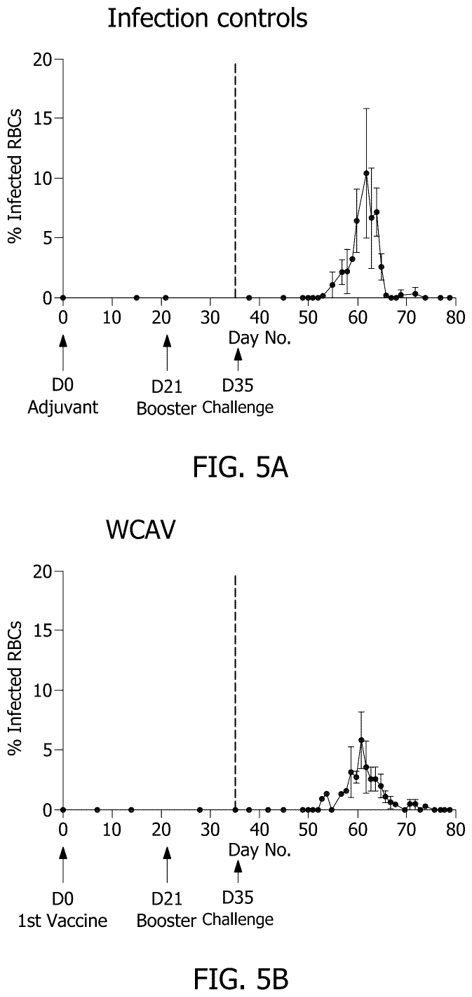

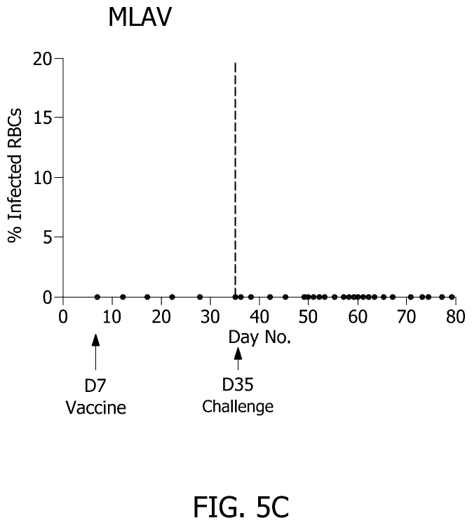

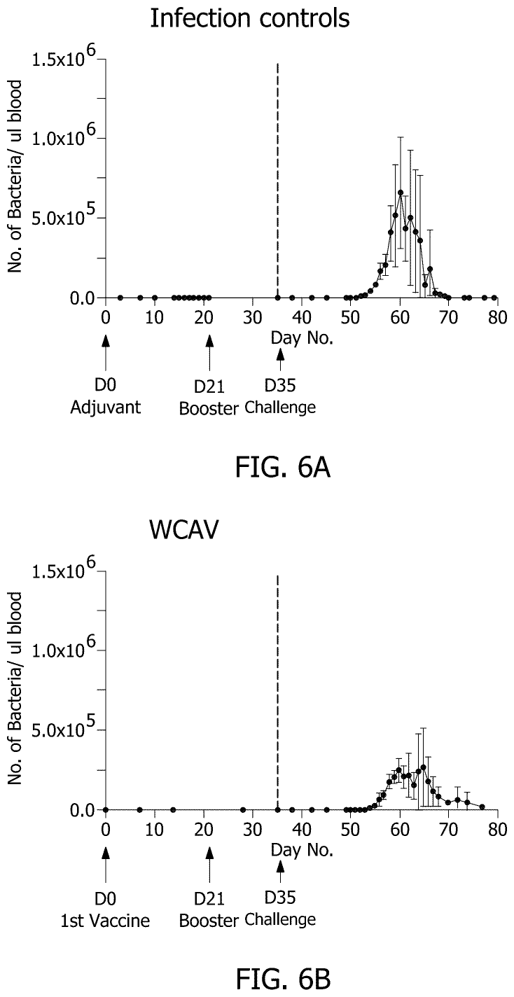

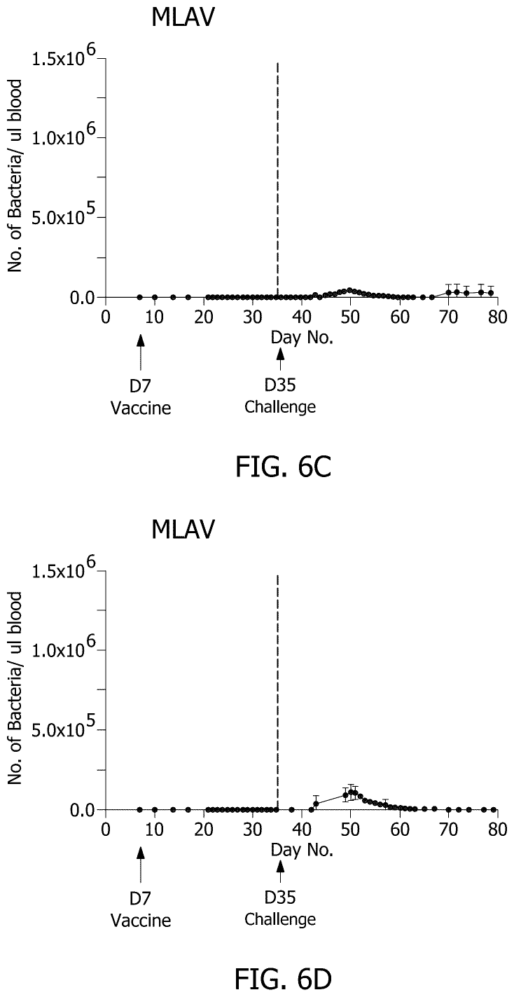

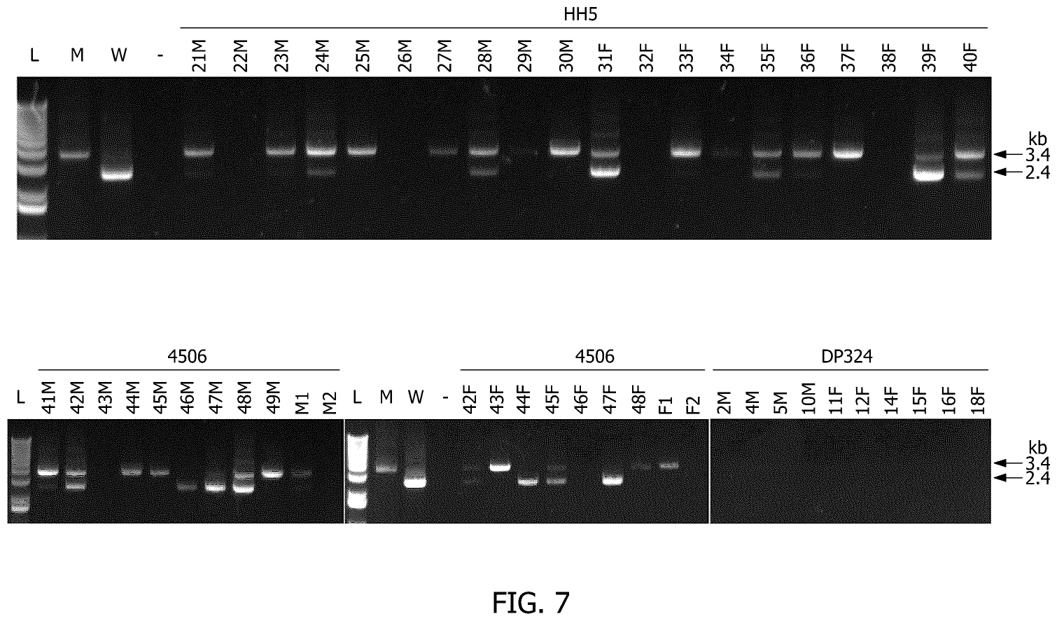

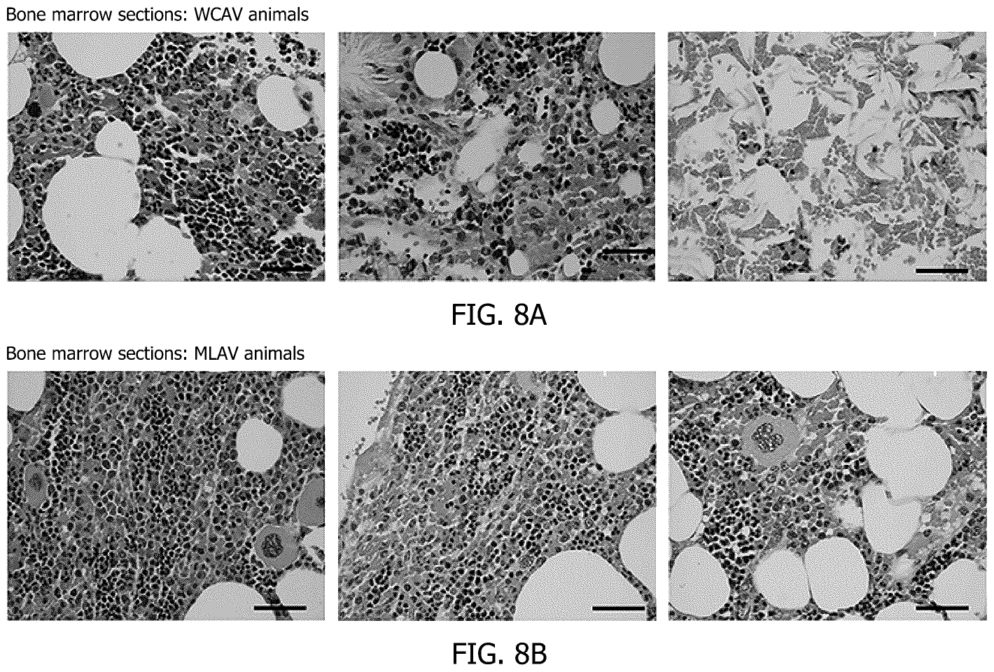

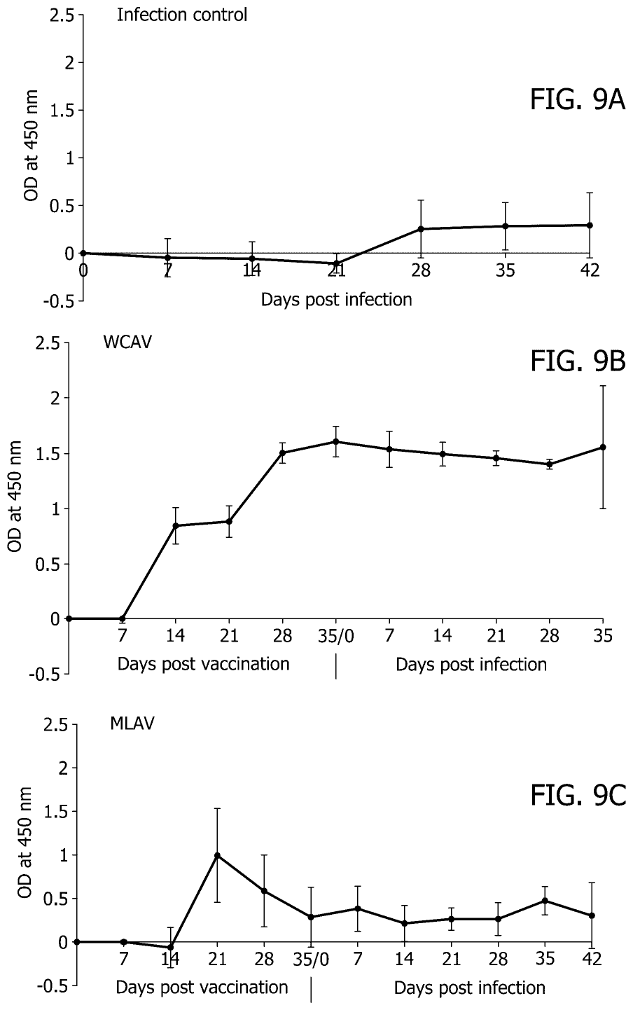

1. Construction of the homologous recombination cassette for use in the A. marginale phtcp gene deletion. Targeted mutagenesis methods that we reported previously for E. chaffeensis were successfully adapted in this study to create a targeted deletion mutation in the A. marginale genome. To generate a gene deletion mutation, 1.1 kb each of A. marginale St. Maries strain (GenBank #CP000030) genomic DNA segments upstream and downstream to the phtcp gene (gene tag #AM581) were engineered to serve as the homology arms in the mutagenesis construct. The fragments were positioned upstream and downstream to mCherry and gentamicin resistance gene coding sequences to be transcribed from the E. chaffeensis tuf promoter. The recombinant plasmid construct (AM581-KO-tuf-mCherry-Gent) was used for the homologous recombination experiments (FIG. 1).
2. Establishing mutagenesis in A. marginale by homologous recombination: The protocol for creating the targeted gene disruption mutation in A. marginale (St. Maries strain) was similar to the method we reported recently for E. chaffeensis (Wang et al., 2017) with a few minor modifications. Gentamicin-resistant A. marginale cultures expressing mCherry were identified after several weeks of assessment in culture (FIG. 2A). PCR assays targeting the upstream and downstream genomic regions used in the allelic exchange and to the inserted mCherry sequence confirmed the presence of the deletion mutation (FIG. 2B). The AM581 gene deletion was further verified by a 3rd PCR assay amplifying the targeted insertion region where a larger amplicon is expected from the mutant compared to that for wild-type (FIG. 2B). Clonal purity of the mutant organisms was further confirmed by Southern blot analysis following digestion of the mutant genomic DNA with HindIII or EcoRV restriction enzymes and hybridized with mCherry gene segment probe. Only the predicted restriction digested fragments were observed in the DNA recovered from the mutant, but not in the genomic DNA recovered from wild-type A. marginale (FIG. 2C).
3. Development of a virulent bovine anaplasmosis infection model: Infection progression in an 18-month-old steer receiving blood stabilate infection was assessed for 92 days by observing for clinical signs, complete blood count (CBC) analysis, and for the presence of infection in erythrocytes by light microscopy. A. marginale erythrocytic inclusions were observed from day 8 post infection which peaked by 29 days to 25% RBCs containing inclusions. The animal also had occasional spikes of temperature. The PCV and hemoglobin levels also steadily declined falling to below normal range by day 28 (22.8% and 7.1 g/dl, respectively) (not shown). Blood was collected on alternate days when the bacteremia levels were about 11% for use in preparing stabilates for infection experiments described below.
4. Impact of A. marginale phtcp gene disruption on in vivo bacterial growth: To test the hypothesis that gene deletion in the A. marginale phtcp gene is detrimental for the pathogen's in vivo growth, approximately 3×10⁸ mutant organisms each were inoculated into cattle intravenously (IV). The steers were monitored for 28 days daily for clinical symptoms, changes in CBC and for the presence of infected erythrocytes. The mutant-inoculated steers did not develop any clinical signs or anemia, and did not exhibit infected erythrocytes detectible by light microscopy. The animals also did not appear to have erythrocyte abnormality; anisocytosis (not shown).
5. Assessment of attenuated mutant and whole cell inactivated antigens as vaccines: We then assessed if the prior inoculation of the A. marginale gene deletion mutant (as a modified live attenuated vaccine; MLAV) confers protection against disease progression resulting from virulent infection challenge. Similarly, we tested inactivated A. marginale whole cell antigens as another vaccine candidate (WCAV). The WCAV was injected twice subcutaneously, after combining with AddaVax oil-in-water emulsion as the adjuvant, on day 0 and day 21. The infection challenges with the virulent A. marginale St. Maries strain were performed on the same day and using the same source of blood stabilate for MLAV, WCAV, and non-vaccinated steers. The non-vaccinated infection control group steers didn't receive either vaccine, however, they were inoculated with the adjuvant as in WCAV steers.
5.1. Complete blood count (CBC) analysis and clinical disease: The non-vaccinated infection controls developed the severe clinical disease, which included occasional spikes of fever, lethargy, inappetence, pale mucous membranes, anemia, and jaundice. In infection control animals, the PCV dropped to as low as 15% (a 57% drop) between days 26-31 post inoculation compared to 35% PCV observed in all groups of steers prior to the infection challenges (FIG. 3A). The disease severity in the infection control group was regarded severe and warranted all three animals required close monitoring by the attending veterinarian with one animal euthanized early on day 31. The WCAV vaccinees also developed similar clinical disease as in non-vaccinated infection control group animals, while the MLAV vaccinees stayed healthy with no detectible clinical signs. The PCV in the WCAV animals also dropped below normal range to 20% (a 43% drop) (FIG. 3B). In contrast, the MLAV animals had a transient mild drop in PCV (25%), but it was within the normal range for steers (FIG. 3C). The PCV levels following the post virulent-infection challenge for the MLAV group animals were significantly higher than the WCAV and non-vaccinated infected group animals for several days of assessment. As with PCV, RBC counts also decreased below normal ranges for the infection controls and WCAV vaccinees, while the MLAV animals had significantly higher RBC counts and they were also within the normal values anticipated for healthy steers (FIG. 3D-F).
5.2 Peripheral blood smear analysis by light microscopy: Blood sampled over several weeks from animals in all three group was assessed for the presence of A. marginale inclusions in erythrocytes by light microscopy (FIGS. 4 and 5); this assay is regarded as a gold standard for monitoring clinical bovine anaplasmosis. Erythrocyte anisocytosis was evident in the blood smears of both the infection controls and WCAV vaccinees, but not in the MLAV vaccinees after about four weeks after infection challenge (FIG. 4A-4C)). The mean percent of infected erythrocytes four weeks following challenge was >10% for the infection control group steers and declined thereafter (FIG. 5A). Similarly, infected erythrocytes were observed in the WCAV vaccinees although the peak infection dropped to about 6% (FIG. 5B). In contrast, the MLAV vaccinees had no detectible infected erythrocytes throughout the 50 days of assessment (FIG. 5C).
5.3. Systemic bacterial copy numbers assessed by quantitative PCR: We then assessed A. marginale DNA copies in blood by a more sensitive real-time quantitative PCR (qPCR) assay targeted to the 16S rDNA gene for the infection controls and WCAV vaccinees (FIG. 6). For the MLAV vaccinees, qPCR was also performed targeting the mutant insertion segment; the mCherry gene. The systemic bacterial loads were the highest for the infection control group, followed by the WCAV vaccinees which peaked by 30 days post infection challenge. The average estimated bacteria at peak were about 7×10⁵ per microliter of blood in non-vaccinated infection control group animals. In the WCAV vaccinees, the bacterial loads were also similarly observed but the peak bacterial numbers were two-thirds less (2.5×10⁵ per microliter of blood). The MLAV group animals had undetectable levels of bacteria prior to virulent infection challenge as per the 16S rDNA qPCR results and similarly, no bacterial numbers were detected also as per the qPCR assay targeting the inserted mCherry gene. Significantly less bacterial numbers were observed as per both the qPCR assay data post infection challenge in the MLAV vaccinees compared to WCAV and non-vaccinate infection control animals (FIG. 6), with peak bacterial numbers detected as <105 per microliter of blood.
5.4. Xenodiagnosis: To further assess the A. marginale infection status in animals post virulent infection challenges, a more sensitive xenodiagnosis assay was performed by allowing D. variabilis nymphal ticks to acquisition feed on the animals. Following blood feeding of nymphs and molting to adults, 20 randomly selected ticks each (10 males and 10 females) per animal (60 total) were tested to determine the presence of A. marginale infection by performing the 16S rDNA gene qPCR. Fifty-nine each out of 60 ticks from both the non-vaccinated infection group and the WCAV group animals were positive for A. marginale. Similarly, ticks from two MLAV group animals tested positive (animal #s HH5 and 4506), while all 2 ticks from the third animal were negative A. marginale infection (animal #DP324) (Table 2). To confirm the absence of A. marginale infection in the third MLAV vaccinee, 40 additional ticks were tested; all of which also tested negative for A. marginale infection (not shown). Most ticks from the two MLAV vaccinees had Ct values within the 2 cycles when comparing between 16S rDNA and mCherry targets, while few ticks had Ct values greater than 2 cycles for the mCherry compared to the 16S rDNA-specific qPCR, suggesting that the ticks harbored both mutant and wild-type bacteria. Thus, we re-evaluated tick DNAs derived from the MLAV vaccinees by a conventional PCR assay targeting the regions upstream to the mutation insertion region with expected size amplicons of 2.4 kb and 3.4 kb for the wild-type and mutant, respectively. We used 20 randomly selected ticks recovered from steers; #HH5 and #4506 and 10 ticks from steer #DP324 (FIG. 7). Ticks recovered from HH5 and 4506 animals were positive primarily for the mutant-specific larger amplicon while a few ticks were positive for the smaller wild-type A. marginale-specific amplicon. Some ticks were negative for both amplicons and similarly few ticks were positive only for the wild-type amplicon. All 10 ticks from steer DP324 were also negative for the conventional PCR.
5.5. Gross and histopathological analysis: All three experimental group animals did not show gross pathology lesions when organs and visceral tissue samples were assessed. Histological examination of bone marrow of the WCAV vaccinees had fewer to no identifiable megakaryocytes, contained cholesterol cleft/atrophy of adipose, edema, and one animal lacked progenitor cells (FIG. 8). Contrary to this, bone marrow from both non-vaccinated infection control animals and MLAV vaccinees were histopathologically unremarkable (FIG. 8).
5.6. Total IgG expression assessed by enzyme-linked immunosorbent assays: A. marginale whole cell antigen-specific IgG response was assessed by ELISA on plasma samples collected at multiple time points post vaccination and post challenge for the WCAV vaccinees and MLAV vaccinees, and post infection challenge for samples collected from non-vaccinated infection controls (FIG. 9). A. marginale-specific IgG response was observed for both the WCAV and MLAV vaccinees, although the response in WCAV animals was greater and increased following booster vaccination; it was about three-fold higher compared to MLAV vaccinees. The IgG levels were significantly lower for the MLAV animal samples compared to WCAV. The non-vaccinated infection controls had the lowest IgG response.

### Discussion:

Targeted mutagenesis in pathogenic bacteria having the ability to inactivate a gene and also to restore a gene function, including tick-transmitted Anaplasmataceae pathogens, is a heavily sought after goal. The disruption mutation in E. chaffeensis phage head to tail connector protein (phtcp) gene (gene tag #ECH_0660) has minimal impact for its in vitro growth, while inducing attenuated growth in two different vertebrate hosts. We reported here that our engineered gene deletion mutation was present only at the intended target site of the A. marginale genome. In this study, we also successfully utilized the mCherry gene and codon-optimized gentamicin resistance gene cassette transcribed from the E. chaffeensis tuf promoter for generating targeted mutations in A. marginale, suggesting that the sequences are broadly applicable for mutagenesis experiments in both Anaplasma and Ehrlichia spp. Previous studies involving Anaplasma spp. reported the use of transposon mutagenesis and it has remained the only option available for creating mutations. The allelic exchange-based targeted mutagenesis will aid in defining genes essential for bacterial pathogenesis in a host, defining host-pathogen interactions, and developing prevention methods for diseases caused by several emerging tick-borne rickettsial diseases. The data presented in the current study extends our prior data reporting that the functional phtcp protein is also critical for A. marginale in vivo growth.

Bovine anaplasmosis continues to cause high economic losses throughout the world resulting from the reduced milk and meat production. Furthermore, the excessive use of tetracycline derivatives added as a food additive for reducing A. marginale infections also contributes to the economic burden and also in increasing the antibiotic resistance risk to animals and humans. Thus, a vaccine to prevent bovine anaplasmosis will be most valuable in both containing the disease and in reducing the antibiotic prophylactic used as a food additive. A live A. centrale blood stabilate vaccine has been in use for several decades in Australia, Israel, and parts of Africa and is regarded the best option in offering heterologous protection against A. marginale infections. Nonetheless, its application is restricted in many countries, such as in the USA, due to the high potential for introducing high risk blood-borne pathogens into cattle. A recent study reported that a live A. marginale strain with a random insertion mutation may serve as a vaccine candidate reducing the disease progression. The data for A. centrale-based heterologous blood stabilate vaccine and a modified live random insertion mutated bacterial vaccine suggest that a bovine anaplasmosis vaccine is likely effective in inducing protective immunity when an attenuated version of the pathogen is used. Indeed, our current study demonstrates that animals receiving one dose of the phtcp gene deletion mutant as a live vaccine offers the best protection in clearing the clinical disease, improving hematological parameters and also in reducing the systemic bacterial loads. On the contrary, the WCAV vaccinees developed clinical disease as the non-vaccinated animals, although some improvements were noted in reducing both the bacterial infection in erythrocytes and anemia. A. marginale was undetectable in MLAV vaccinees in erythrocytes when assessed by light microscopy and lacked anemia. A more sensitive qPCR assay demonstrated the presence of both the mutant and wild-type A. marginale in the blood of MLAV vaccinees although the bacterial numbers were significantly lower compared to WCAV vaccinees and non-vaccinated animals. Further, xenodiagnosis substantiated the presence of low-level circulation of the mutant and wild-type A. marginale. The infection-persistence, however, was observed in only two of the three MLAV vaccinees. The data suggest that despite the absence of clinical disease and recovery from anemia, the MLAV did not offer complete sterile immunity at least in two of the three animals assessed.

The bone marrow was normal in MLAV vaccinees, thus the vaccine also helped to keep the bone marrow healthy as in comparison to non-vaccinated animals. It is unclear why WCAV vaccinees had the loss of megakaryocytes in bone marrow, and other changes, such as adipocyte atrophy, cholesterol clefts, and edema. One possible explanation is that the vaccine-induced immunity in WCAV vaccinees may have adversely impacted animals when receiving the virulent pathogen challenge. Modified live vaccines are likely to activate all arms of the immune system and provide immunity to combat clinical disease. We reported previously that E. chaffeensis attenuated mutant with the phtep gene mutant as the live vaccine provided complete protection for dogs against virulent pathogen infection challenge by IV inoculation and by tick transmission. The current study assessed only IV infection challenge with a homologous virulent strain of A. marginale. Live A. centrale blood stabilate vaccine is generally regarded as having the ability to confer protection against A. marginale infections by both mechanical and tick-transmission challenge. Thus, A. marginale phtcp gene deletion mutant as a live vaccine will offer sufficient protection against the disease resulting from diverse A. marginale strains transmitted from ticks and by mechanical transmission. Induction of T cell responses during intracellular bacterial infections is known to play a greater role in generating protection against infection than B cell responses. Consistent with the previous observations, higher antibody response observed in the WCAV vaccinates did not aid in preventing the clinical disease, neither in reducing infection in erythrocytes nor in restoring the loss of erythrocytes. Protective response against bovine anaplasmosis, therefore, is more than just the induction of the B cell response; the present study is the critical first step in furthering studies to define the immune mechanisms of protection. The study is also important in determining if MLAV offers protection against diverse A. marginale strains transmitted mechanically or from an infected tick.

### Example 2

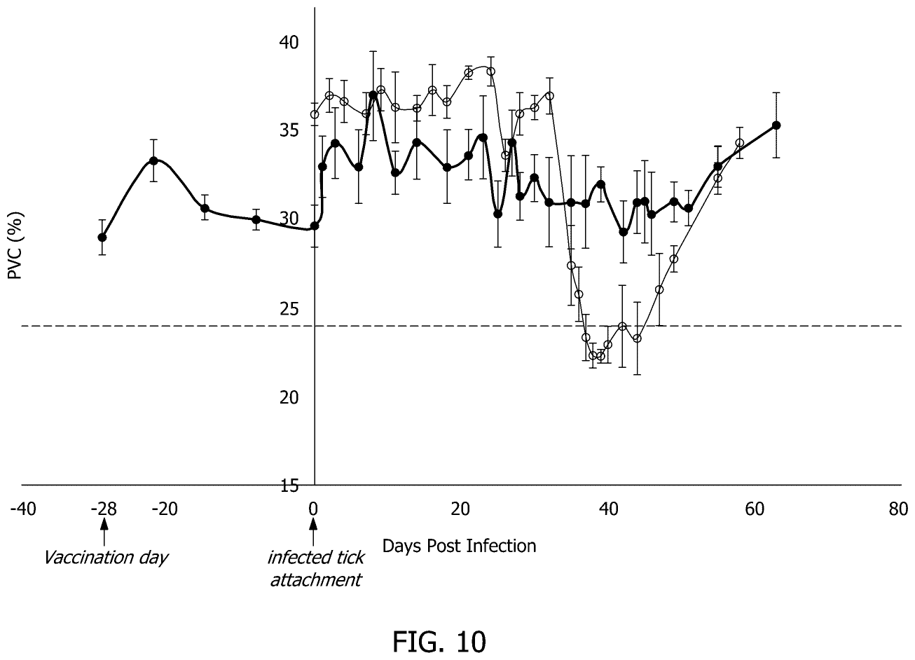

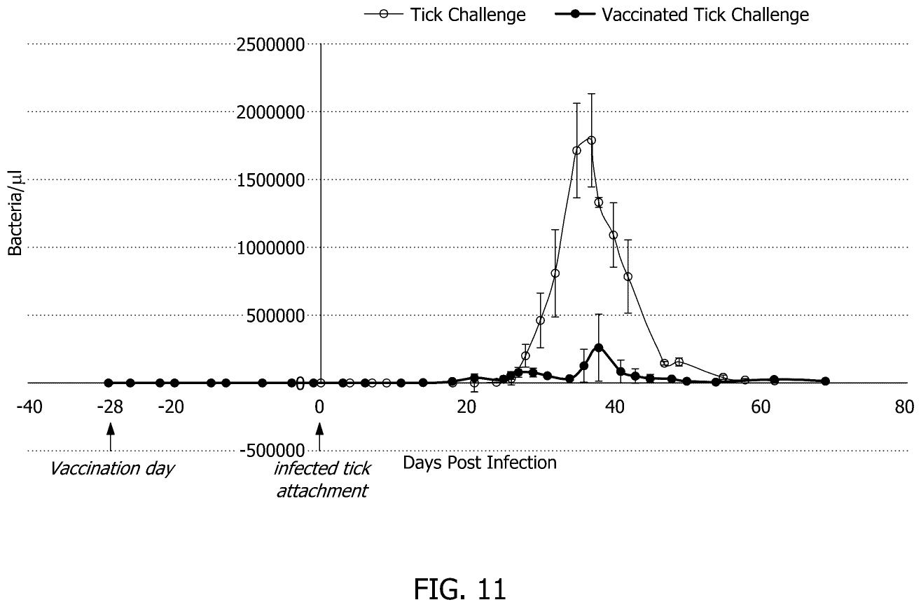

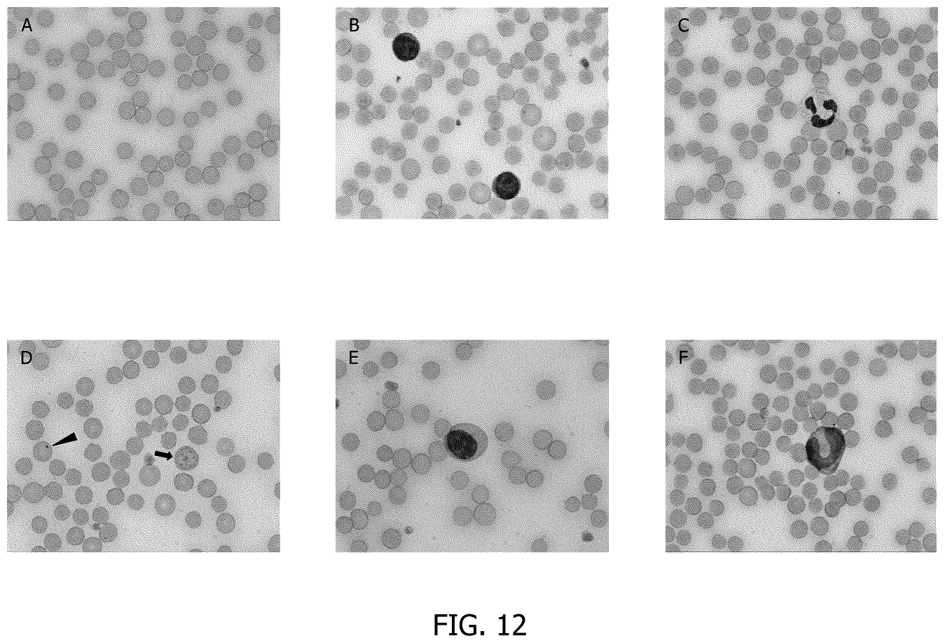

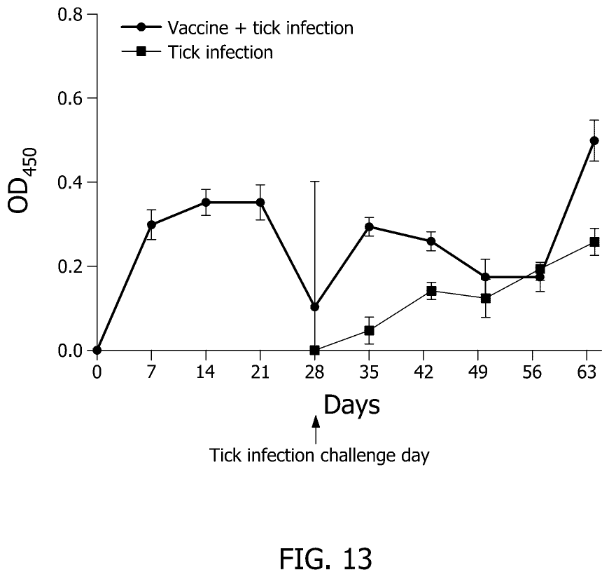

MLAV also prevented bovine anaplasmosis resulting from tick transmission. To assess if the MLAV similarly protects against tick transmission challenge, we performed another vaccine study where we included two groups of animals (n=3). Initially, male Dermacentor variabilis ticks were allowed to acquisition feed on cattle infected with a wildtype A. marginale during peak bacteremia to generate infected ticks. Following a week of blood feeding, all ticks were removed and held in a 25° C. humidified incubator for 5-13 days prior to using them for infection transmission feeding experiments. A. marginale infection status in the ticks was confirmed by qPCR; 94% of ticks (16 of the 17 ticks) tested positive for the presence of the pathogen. Three cattle were vaccinated with MLAV, while three nonvaccinated steers were kept as infection controls. Ticks were placed on vaccinated animals four weeks following the vaccination. Infected D. variabilis males (44 ticks per animal) were then allowed to feed on each animal for a week. After this time, animals from both the nonvaccinated and vaccinated groups were monitored for clinical signs and infection. Blood was sampled over 70 days to monitor changes in the blood cell abnormalities and for the infection status by qPCR. Nonvaccinated animals developed severe clinical disease exhibiting high fever, lethargy, and inappetence consistent with anaplasmosis. A drop in the PCV to ˜23% from the normal range of ˜35% (about 34% decline) was observed in these cattle after day 32 post infected tick attachment and all three animals in this group remained anemic for several days (FIG. 10). During this time, the bacteremia peaked to ˜1.75×106 per microliter of blood (FIG. 11). Significantly more blood cell abnormalities were evident in the nonvaccinated tick-transmission animals, which included anisocytosis, reticulocytosis, activated lymphocytes, and band cells (FIG. 12 and Table 3 below). In contrast, MLAV vaccinated cattle remained asymptomatic both before and after tick transmission challenge. The vaccinated animals also had normal PCV values, with a minimal presence of bacteremia (a 7-fold drop) and had predominantly healthy blood cells. As observed against I.V. infection challenge protection experiments, antibody levels were similar in cattle from both nonvaccinated and vaccinated animals (FIG. 13).

TABLE 3
Blood film observations from throughout the bacteremia
phase of the study; days 21-49 (8 time points)

| | | | Activated | Band |
|---|---|---|---|---|
| | Reticulocytosis | Anisocytosis | Lymphocytes | Cells |
| Tick | 12 (50%) | 16 (67%) | 19 (79%) | 17 (71%) |
| Transmission | | | | |
| Control | | | | |
| Vaccinated | 1 (4%) | 7 (29%) | 8 (33%) | 7 (29%) |
| Group | | | | |

The data demonstrate that the modified live attenuated vaccine provides sufficient immune protection against both needle infection (mechanical transmission) and tick transmission of virulent A. marginale.

What is claimed is:

1. An immunogenic composition comprising an Anaplasma marginale (A. marginale) having a gene disruption or deletion in the genome thereof.

2. The immunogenic composition of claim 1, wherein the gene disruption is in the phtcp gene.

3. The immunogenic composition of claim 1, wherein the phtcp gene is deleted from the A. marginale genome.

4. The immunogenic composition of claim 1, further comprising at least one additional element selected from the group consisting of an additional antigen, pharmaceutical-acceptable carrier, diluent, veterinary-acceptable carrier, adjuvant, preservative, stabilizer, or any combination thereof.

5. The immunogenic composition of claim 4, wherein the additional antigen is from a pathogen selected from the group consisting of Actinobacillus pleuropneumonia; Adenovirus; Alphavirus such as Eastern equine encephalomyelitis viruses; Bordetella bronchiseptica; Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl. septicum, Cl. tetani, Coronavirus, preferably Porcine Respiratory Corona virus; Eperythrozoonosis suis; Erysipelothrix rhusiopathiae; Escherichia coli; Haemophilus parasuis, preferably subtypes 1, 7 and 14: Hemagglutinating encephalomyelitis virus; Japanese Encephalitis Virus; Lawsonia intracellularis; Leptospira spp.; preferably Leptospira australis, Leptospira canicola; Leptospira grippotyphosa, Leptospira icterohaemorrhagicae; and Leptospira interrogans; Leptospira pomona, Leptospira tarassovi; Mycobacterium spp. preferably M. avium; M. intracellulare; and M. bovis; Mycoplasma hyopneumoniae (M hyo); Pasteurella multocida; Porcine cytomegalovirus; Porcine Parvovirus; Porcine Reproductive and Respiratory Syndrome (PRRS) Virus; Porcine circovirus, Pseudorabies virus; Rotavirus; Salmonella spp.; preferably S. thyhimurium; and S. choleraesuis; Staph. hyicus; Staphylococcus spp., Streptococcus spp., preferably Strep. suis; Swine herpes virus; Swine Influenza Virus; Swine pox virus; Swine pox virus; Vesicular stomatitis virus; Virus of vesicular exanthema of swine; Leptospira Hardjo; Mycoplasma hyosynoviae; Poliovirus; Rhinovirus; hepatitis A virus; foot-and-mouth disease virus (FMDV); swine vesicular disease (SVDV), and any combination thereof.

6. The immunogenic composition of claim 4, wherein said adjuvant is an emulsion.

7. The immunogenic composition of claim 4, wherein said diluent is selected from the group consisting of water, saline, dextrose, ethanol, glycerol, and any combination thereof.

8. A method of reducing the incidence of, severity of, or duration of signs of A. marginale infection comprising the steps of administering at least one dose of the immunogenic composition of claim 1 to an animal in need thereof.

9. The method of claim 8, wherein said immunogenic composition is administered intravenously, intramuscularly, intranasally, intradermally, intratracheally, intravaginally, intravenously, intravascularly, intraarterially, intraperitoneally, orally, intrathecally, or by direct injection into any target tissue.

10. The method of claim 8, wherein said incidence of, severity of, or duration of signs of A. marginale infection is reduced by at least 10% in an animal or group of animals in comparison to an animal or group of animals that did not receive at least one administration of the immunogenic composition.

11. The method of claim 8, wherein said immunogenic composition is administered more than one time and said administrations are separated by at least 2 weeks.

12. The method of claim 8, wherein said immunogenic composition is administered prior to infection by A. marginale.

13. The method of claim 8, wherein said immunogenic composition is administered to an animal in need thereof that is at least 2 weeks of age.

14. The method of claim 8, wherein said immunogenic composition is administered after said animal in need thereof has been exposed to A. marginale.

15. The method of claim 8, wherein said sign of A. marginale infection is selected from the group consisting of anemia, fever, abortion, pale mucous membranes, jaundice, weight loss, poor production, decrease in weight gain, death, anaplasmosis, weakness, pallor, lethargy, dehydration, anorexia, pale tissues, decreased packed cell volume, watery blood, thin blood, splenomegaly, hepatomegaly, gall bladder distension, membrane-bound inclusions (colonies) in the cytoplasm of infected erythrocytes, loss of megakaryocytes in bone marrow, adipocyte atrophy, cholesterol clefts, edema, hemolysis, and any combination thereof.

16. A method of decreasing the transmissibility of A. marginale infection from a host to a vector comprising the step of administering at least one dose of the immunogenic composition of claim 1 to an animal in need thereof.

17. The method of claim 16, wherein said transmissibility is reduced at least 10% in comparison to an animal that did not receive an administration of said immunogenic composition.

18. The method of claim 16, wherein said immunogenic composition is administered intravenously, intramuscularly, intranasally, intradermally, intratracheally, intravaginally, intravenously, intravascularly, intraarterially, intraperitoneally, orally, intrathecally, or by direct injection into any target tissue.

19. The method of claim 16, wherein said immunogenic composition is administered more than one time and said administrations are separated by at least 2 weeks.

20. The method of claim 16, wherein said immunogenic composition is administered prior to infection by A. marginale.

21. The method of claim 16, wherein said immunogenic composition is administered to an animal in need thereof that is at least 2 weeks of age.

22. The method of claim 16, wherein said immunogenic composition is administered after said animal in need thereof has been exposed to A. marginale.

23. A method of making an immunogenic composition for reducing the incidence, severity, or duration of signs of A. marginale infection comprising the step of deleting or disrupting the pthcp gene of a strain of A. marginale to produce a mutant A. marginale.

24. The method of claim 23, wherein the entire phtcp gene is deleted.

25. The method of claim 23, wherein said phtcp gene normally expresses a protein having at least 90% sequence homology with SEQ ID NO. 21.

26. The method of claim 23, wherein said mutant A. marginale is combined with at least one additional element selected from the group consisting of an additional antigen, pharmaceutical-acceptable carrier, diluent, veterinary-acceptable carrier, adjuvant, preservative, stabilizer, or any combination thereof.

27. The method of claim 26, wherein the additional antigen is from a pathogen selected from the group consisting of Actinobacillus pleuropneumonia; Adenovirus; Alphavirus such as Eastern equine encephalomyelitis viruses; Bordetella bronchiseptica, Brachyspira spp., preferably B. hyodyentheriae; B. piosicoli, Brucella suis, preferably biovars 1, 2, and 3; Classical swine fever virus; Clostridium spp., preferably Cl. difficile, Cl. perfringens types A, B, and C, Cl. novyi, Cl. septicum, Cl. tetani; Coronavirus, preferably Porcine Respiratory Corona virus; Eperythrozoonosis suis; Erysipelothrix rhusiopathiae; Escherichia coli; Haemophilus parasuis, preferably subtypes 1, 7 and 14: Hemagglutinating encephalomyelitis virus; Japanese Encephalitis Virus; Lawsonia intracellularis; Leptospira spp.; preferably Leptospira australis; Leptospira canicola; Leptospira grippotyphosa, Leptospira icterohaemorrhagicae; and Leptospira interrogans; Leptospira pomona, Leptospira tarassovi; Mycobacterium spp. preferably M. avium; M. intracellulare; and M. bovis; Mycoplasma hyopneumoniae (M hyo); Pasteurella multocida, Porcine cytomegalovirus; Porcine Parvovirus; Porcine Reproductive and Respiratory Syndrome (PRRS) Virus; Porcine circovirus, Pseudorabies virus; Rotavirus; Salmonella spp.; preferably S. thyhimurium; and S. choleraesuis; Staph. hyicus; Staphylococcus spp., Streptococcus spp., preferably Strep. suis; Swine herpes virus; Swine Influenza Virus; Swine pox virus; Swine pox virus; Vesicular stomatitis virus; Virus of vesicular exanthema of swine; Leptospira hardjo; Mycoplasma hyosynoviae; Poliovirus; Rhinovirus; hepatitis A virus; foot-and-mouth disease virus (FMDV); swine vesicular disease (SVDV), and any combination thereof.

28. The method of claim 26, wherein said adjuvant is an emulsion.

29. The method of claim 26, wherein said diluent is selected from the group consisting of water, saline, dextrose, ethanol, glycerol, and any combination thereof.
